(19) United States
(12) Patent Application Publication
(10) Pub. No.: US 2026/0243765 A1
(43) Pub. Date: Aug. 20, 2026

(54) METHOD FOR DETECTING HPV INFECTION IN NON-EROSIVE LICHEN PLANUS

(71) Applicants: INSTITUT PASTEUR, PARIS CEDEX 15 (FR); INSTITUT NATIONAL DE LA SANTE ET DE LA RECHERCHE MEDICALE, PARIS (FR); UNIVERSITE PARIS CITE, PARIS (FR); UNIVERSITE DE REIMS CHAMPAGNE-ARDENNE, REIMS (FR); ASSISTANCE PUBLIQUE HOPITAUX DE PARIS, PARIS (FR)

(72) Inventors: Marie-Lise GOUGEON, PARIS (FR); Manuelle VIGUIER, REIMS (FR); Nicolas FAZILLEAU, LA SALVETAT SAINT GILLES (FR); Herve BACHELEZ, PARIS (FR)

(21) Appl. No.: 18/992,749

(22) PCT Filed: Jul. 10, 2023
(86) PCT No.: PCT/EP2023/069088

§ 371 Date: Jan. 9, 2025

Related U.S. Application Data
(60) Provisional application No. 63/359,803, filed on Jul. 9, 2022.
(60) Provisional application No. 63/376,006, filed on Sep. 16, 2022.

(51) Int. Cl. G01N 33/569; G01N 33/50

(52) U.S. Cl. CPC G01N 33/56972; G01N 33/505; G01N 33/56983; G01N 2333/025; G01N 2469/10

(57) ABSTRACT

The present invention relates to Lichen planus (LP), and more specifically to no erosive form of LP (NELP). The invention provides an immune signature for LP, which is not found in other cutaneomucosal chronic inflammatory diseases. The invention moreover provides different diagnosis methods and methods of treating NELP, based on this immune signature. The inventors have indeed demonstrated the presence of HPV16-specific activated CTL in blood and lesion samples corresponding to the different clinical forms of LP, not only in erosive oral LP, while a different scenario operates in other cutaneomucosal chronic inflammatory diseases. This HPV16-specific activated CTL is characterized by TCRVβ3+ CD8+ T-cells oligoclonal expansions.

### CROSS-REFERENCE TO RELATED APPLICATIONS

This application is the U.S. Natl. Stage of international application PCT/EP2023/069088, which claims the benefit of U.S. Provisional Application 63/359,803 filed on Jul. 9, 2022, and U.S. Provisional Application 63/376,006 filed on Sep. 16, 2022.

### REFERENCE TO SEQUENCE LISTING SUBMITTED ELECTRONICALLY

The instant application contains a Sequence Listing which has been submitted electronically in XML format and is hereby incorporated by reference in its entirety. Said XML copy, created on Jan. 31, 2025, is named B2301363WOUS.xml and is 53,343 bytes in size.

### BACKGROUND OF THE INVENTION

Lichen planus (LP) is a chronic inflammatory disease of unknown etiology, involving the skin and mucous membranes, characterized by an infiltrate of immune cells, mainly CD8⁺ cytotoxic T-lymphocytes (CTL), associated with epithelial cell death and disruption of basement membrane zone, as pyramidal features (Zhou et al., 2002). Numerous clinical and evolutive forms are described, as well as different locations (palms, soles, hair, nails, oral, genitals) (Le Cleach and Chosidow, 2012), all sharing the same pathophysiological features with a cell-mediated immune damage of the basal keratinocytes potentially through the recognition of foreign antigens. Analysis of LP skin biopsies indicated involvement of type I IFNs, associated with the recruitment of CXCR3⁺ and granzyme B⁺ lymphocytes, indicating a Th1-biased cytotoxic immune response (Wenzel et al., 2006).

Several triggering factors have been advocated in the mechanistic scenario in LP, including drugs, metals, bacterial or viral antigens. Among viral candidates, the association of LP with hepatitis B virus (HBV) infection, suspected as soon as three decades ago (Rebora and Rongioletti, 1984) and reinforced by the description of LP onset after HBV vaccination (Ciaccio and Rebora, 1990; Calista and Morri, 2004), was ultimately ruled out by several large case-control studies conducted in different countries with high HBV prevalence (Birkenfeld et al., 2011; Jayavelu and Sambandan, 2012; Song et al., 2016). For hepatitis C virus (HCV) infection, several meta-analyses demonstrated a significant association between HCV infection and oral LP (Shengyuan et al., 2009; Lodi et al., 2010; Alaizari et al., 2016), with clinical benefit of HCV antiviral drugs on LP (Nagao et al., 2017). More recently, viral species with known mucosal and/or skin tropism, such as Epstein Barr virus (EBV), Herpes simplex virus and Human Papillomavirus virus (HPV), attracted special interest (Lodi et al., 2005). Regarding HPV, a meta-analysis of published case-control studies showed that patients with oral LP had a significantly higher HPV detection in oral mucosa, by either PCR, immunohistochemical staining or in situ hybridization methods, than healthy controls, with a strong association with HPV16 genotype and higher rates of detection in erosive versus non-erosive oral LP lesions (Ma et al., 2016; Shang et al., 2020).

Altogether, these epidemiological associations, as well as the T-cell signature of LP, raised the hypothesis of a mechanistic link bridging HPV-mucosal and cutaneous infection and cytotoxic T-cell responses in LP. Tackling this hypothesis, the inventors recently showed that patients with erosive oral LP share oligoclonal expansions of HPV16-specific CTL in both the peripheral blood and the epithelial lesions, suggesting a key contribution of HPV16-specific CTL to the pathogenesis of erosive oral LP (Viguier et al., 2015). Here, the inventors address the question of whether this immunological pattern is specific to erosive-LP, especially severe erosive ones, or is also observed in other clinical forms of LP, such as non-erosive LP, (NELP) where the CTL contribution appears less decisive, and in a dermatosis also affecting the genital mucosa, the lichen sclerosus et atrophicus (LSA).

### BRIEF SUMMARY OF THE INVENTION

Lichen planus is a chronic inflammatory disease of unknown etiology, involving the skin and mucous membranes, characterized by infiltrates of CD8⁺ cytotoxic T-lymphocytes, associated with epithelial cell death and disruption of basement membrane zone. The inventors previously showed that patients with erosive oral lichen planus display oligoclonal expansions of Human Papilloma Virus (HPV)16-specific cytotoxic T-lymphocytes in both the peripheral blood and the epithelial lesions. Herein, they investigated whether a similar scenario operates in other clinical forms of lichen planus, such as non-erosive LP which is a distinct pathology, and in lichen sclerosus et atrophicus, another chronic disease also affecting the mucosa and/or the skin. Unexpectedly, oligoclonal expansions of CD8⁺ TCRVβ3⁺ T-lymphocytes with increased frequency of HPV16-specific CD8⁺ T-cells were also detected in the blood and in the skin of patients with different forms of lichen planus, irrespective of its erosive nature, and thus also in non-erosive LP, but not observed in lichen sclerosus and atrophicus. Thus, the inventors identified an immune signature characteristic of lichen planus, both erosive LP and non-erosive LP, which are 2 distinct pathologies, that could be used in clinical settings in the future: first of all, to confirm lichen planus diagnosis, irrespective of its erosive nature or severity of symptoms, when clinical and pathological features are not sufficient, and, moreover, to develop new therapeutic approaches by deleting or anergizing pathogenic T-cells through the immunotargeting of the pathogenic CD8⁺TCRVβ3⁺ population.

The invention encompasses compositions and methods for the diagnosis and treatment of LP patients, preferably non-erosive LP patients, LP patients without oral erosive LP or LP patients without oral LP. The invention encompasses a method for detecting the presence of an immune response against a human papilloma virus infection in a biological sample, especially a blood sample or lesional sample of a suspected non-erosive lichen planus (NELP) patient, preferably one with a lichen planus which is not an oral lichen planus, said method comprising isolating T cells from a biological sample from the patient to be diagnosed, preferably one with a suspected lichen planus which is not an oral lichen planus, and detecting expansion of a clonal population of CD8+ TCRVbeta3+ T cells recognizing the HPV16 E713-24 epitope in the sample, wherein said clonal population has a single CDR3beta sequence in the TCRVbeta3 gene segment.

In one embodiment, the CDR3beta sequence in the TCRVbeta3 gene segment in the CD8+ T cells encodes any one of the following peptides: SRTVNTI (SEQ ID NO:1), SLVETGFIGTTFEVEQ (SEQ ID NO:2), SLGVGYEQ (SEQ ID NO:3), SLRGAYEQ (SEQ ID NO:4), SFQGFETQ (SEQ ID NO:5), SLSTPNYEQ (SEQ ID NO:6), SFQGYYEQ (SEQ ID NO:7), SWTRTYNEQ (SEQ ID NO:8), SFGLRAGTYEQ (SEQ ID NO:9), SLTSATGEL (SEQ ID NO:10), SLRGAINGEL (SEQ ID NO:11), SLWAGNLREQ (SEQ ID NO:12), a consensus peptide consisting of: S in position 1, L, V or F in position 2, G or R in position 3, V or G in position 4, G, A or H in position 5, Y in position 6, E in position 7, and Q in position 8 (SEQ ID NO:60).

In one embodiment, the method further comprises preparing nucleic acids from the biological sample, e.g. blood or legional sample and contacting the nucleic acids with an HPV specific primer or probe.

In one embodiment, the method further comprises staining PMBC of the blood sample or lesional skin biopsy from the NELP patient or suspected NELP patient with peptide-containing MHC class I dextramer specific for E713-24 immunodominant peptide of HPV16.

In one embodiment, the method further comprises spectratyping analysis of the blood sample or of a lesional skin biopsy from the NELP patient, or suspected NELP patient, with TCRVbeta specific primers.

In one embodiment, the NELP patient or suspected NELP patient, has a lichen planus which is not an oral lichen planus. In one embodiment, the NELP patient has a skin lichen planus. In one embodiment, the NELP patient has a scalp lichen planus. In one embodiment, the NELP patient has a nail lichen planus. In one embodiment, the non-oral LP is a genital LP. The sample is thus accordingly a skin sample, a scalp sample or a genital sample.

The method encompasses a method for detecting the presence of a clonal expansion of CD8+ T cells in a biological sample, e.g. blood sample or lesional sample, of a patient suspected to have a lichen sclerosus et atrophicus (LSA), said method comprising isolating T cells from a blood sample from the patient and detecting expansion of a clonal population of CD8+ TCRVbeta6+ T cells in the sample, wherein said clonal population has a single CDR3beta sequence in the TCRVbeta6 gene segment.

In one embodiment, the CDR3beta sequence in the TCRVbeta6 gene segment in the CD8+ T cells encodes any one of the following peptides: SSMGVTEA (SEQ ID NO: 48), SFWQVPGEL (SEQ ID NO: 49), SPYRSSYNEQ (SEQ ID NO: 50), SSTSWGETQ (SEQ ID NO: 51), SLGRGPGDTQ (SEQ ID NO: 52), SHQTDEA (SEQ ID NO: 53), STRTGRFEKL (SEQ ID NO: 54), SSAGQGYTEA (SEQ ID NO: 55), SSPTGGLDEAFF (SEQ ID NO: 56), SPPTGDYEQYF (SEQ ID NO: 57), SSNTASSYNEQ (SEQ ID NO: 58), SNSTSGYGY (SEQ ID NO: 59).

The invention encompasses a method for monitoring treatment of an LP patient, preferably of an NELP patient. In one embodiment, the method comprises providing a cell sample, preferably a lesion or a blood sample from an LP patient, and detecting the presence of an immune response against a human papilloma virus infection in the cell sample. In one embodiment, the method comprises providing a cell sample, preferably a lesion or a blood sample from an LP patient, and detecting the presence of a human papilloma virus. In one embodiment, the method further comprises treating the patient with an anti-HPV treatment, and, optionally, detecting a reduction in an LP symptom. In another embodiment, the method further comprises treating the patient with corticosteroids, retinoids, immunosuppressants, or phototherapy, and, optionally, detecting a reduction in an LP symptom.

Throughout this specification, an LP patient has preferably a non-erosive lichen planus. In another embodiment, the patient has a lichen planus which is not an oral LP form. According to still another embodiment, the patent has a non-erosive LP which is not an oral LP form. Throughout this specification, an “LP patient” means the patient has a confirmed lichen planus (disease) or is suspected to have a lichen planus (disease).

In some embodiments the human papilloma virus is of a type selected from types 16, 18, 31, 33, 35, 39, 45, 51, 52, 56, 58, 59, 68, 69, 73, and 82.

In some embodiments the anti-HPV treatment is selected from a composition comprising the major capsid protein L1 of HPV types 6, 11, 16, and 18; a composition comprising the major capsid protein L1 of HPV types 16, and 18; and at least one chimeric recombinant Bordetella sp. adenylate cyclase (CyaA) protein or fragment thereof, the CyaA protein or fragment thereof comprising at least one inserted human papilloma virus (HPV) E7 epitope.

In one embodiment, the presence of an immune response against a human papilloma virus infection is detected by contacting the sample with at least one HPV peptide, more particularly at least one E7 HPV peptide, preferably at least one peptide comprising the amino acid sequence YMLDLQPETT (SEQ ID NO:33).

In one embodiment, the presence of an immune response against a human papilloma virus infection is detected by sequencing the CDR3β sequence of the TCRVβ3 gene segment in the TCR of T cells of the cell sample. Preferably, the CDR3β sequence of the TCRVβ3 gene segment in the T cells of the cell sample comprises any one of the CDR3 peptide sequences SEQ ID NOs 1-12.

In one embodiment, the presence of an immune response against a human papilloma virus infection is detected by detecting a clonal population of CD8+TCRVβ3+ T cells in the biological sample or cell sample.

In one embodiment, the presence of a human papilloma virus is detected by preparing nucleic acids from the biological or cell sample and contacting the nucleic acids with at least one HPV specific primer or probe.

The invention encompasses a method for detecting an HPV infection, or past infection, in an NELP patient comprising providing a non-oral cell sample from the NELP patient, contacting the non-oral sample with at least one HPV peptide, and detecting the interaction between the HPV peptide and cells in the sample. The sample is preferably a sample of the LP lesion.

In one embodiment, the HPV peptide comprises the amino acid sequence YMLDLQPETT (SEQ ID NO:33). In one embodiment, the non-oral sample is a skin sample. In one embodiment, the non-oral sample is a scalp sample. In one embodiment, the non-oral sample is a nail sample. In one embodiment, the non-oral sample is a genital sample.

The invention encompasses a method for detecting an HPV infection in an NELP patient comprising providing a non-oral cell sample from the NELP patient, preparing nucleic acids from the non-oral cell sample, contacting the nucleic acids with an HPV specific primer or probe, and detecting the interaction between the HPV specific primer or probe and the nucleic acids from the non-oral cell sample.

The invention encompasses methods for diagnosing an LP patient or suspected LP patient, preferably an NELP patient. In one embodiment, the method comprises providing a cell sample or a biological sample, preferably a lesion or a blood sample, from an LP patient, especially a patient suspected of having an LP and preferably a NELP, especially a skin, scalp, genital sample, or nail sample, contacting the sample with at least one HPV peptide; and detecting the interaction between the HPV peptide and cells in the sample. Preferably, the HPV peptide comprises the amino acid sequence YMLDLQPETT (SEQ ID NO:33).

The method can comprise preparing nucleic acids from the cell sample and contacting the nucleic acids with an HPV specific primer or probe.

The methods of the invention can comprise treating the patient with an anti-HPV treatment, or with an LP-treatment, such as corticosteroids, retinoids, immunosuppressants, or phototherapy, providing a second cell sample from an LP patient, contacting the sample with at least one HPV peptide; and detecting the interaction between the HPV peptide and cells in the sample.

In one embodiment, the method comprises providing a blood sample from an LP patient, preferably an NELP patient, isolating T cells from the blood sample, and detecting CDR3β distribution of the TCRVβ3 gene segment in the T cells.

The method can comprise treating the patient with an anti-HPV treatment, preferably cidofovir, and providing a second blood sample from an LP patient, isolating T cells from the blood sample, and detecting CDR3β distribution of the TCRVβ3 gene segment in the T cells. The method can comprise preparing nucleic acids from the blood sample and contacting the nucleic acids with an HPV specific primer or probe.

In one embodiment, the method comprises providing a blood sample from an LP patient, isolating T cells from the blood sample, and determining the CDR3β sequence of the TCRVβ3 gene segment in the T cells. Preferably, the CDR3β sequence of the TCRVβ3 gene segment in the T cells of the cell sample comprises any one of SEQ ID Nos 1-12.

The method can comprise preparing nucleic acids from the cell sample and contacting the nucleic acids with an HPV specific primer or probe.

The invention encompasses methods for treating an LP patient. In one embodiment, the method comprises providing a biological sample, preferably a blood sample or lesional sample, from an LP patient, isolating T cells from the biological sample, detecting a clonal population of CD8+TCRVβ3+ T cells, and treating the patient with a compound specific for the CD8+TCRVβ3+ T cells. In one embodiment, the patient has a non-erosive lichen planus. In another embodiment, the patient has a lichen planus which is not an oral LP form. According to still another embodiment, the patent has a non-erosive LP which is not an oral LP form.

The invention encompasses a method comprising treating the patient with (i) an anti-HPV treatment, and/or (ii) a steroid treatment, corticosteroids, retinoids, immunosuppressant treatment, or phototherapy or photochemotherapy.

The invention encompasses the use of a compound specific for CD8+TCRVβ3+ T cells for the treatment of LP, preferably of non-erosive lichen planus, or LP which not oral LP, or non-erosive LP which are not oral LP.

The invention also encompasses a method for treating an NELP patient comprising selecting an HPV positive NELP patient; and treating the HPV positive NELP patient with extracorporeal photochemotherapy (ECP). In some embodiments the human papilloma virus is a high-risk HPV virus. In some embodiments the human papilloma virus is of a type selected from types 16, 18, 31, 33, 35, 39, 45, 51, 52, 56, 58, 59, 68, 69, 73, and 82.

The invention also encompasses a method for treating an NELP patient comprising selecting an HPV positive NELP patient; and treating the HPV positive NELP patient with a treatment selected from a composition comprising the major capsid protein L1 of HPV types 6, 11, 16, and 18; a composition comprising the major capsid protein L1 of HPV types 16, and 18; and at least one chimeric recombinant Bordetella sp. Adenylate cyclase (CyaA) protein or fragment thereof, the CyaA protein or fragment thereof comprising at least one inserted human papilloma virus (HPV) E7 epitope. In some embodiments the human papilloma virus is a high risk HPV virus. In some embodiments the human papilloma virus is of a type selected from types 16, 18, 31, 33, 35, 39, 45, 51, 52, 56, 58, 59, 68, 69, 73, and 82.

### BRIEF DESCRIPTION OF THE DRAWINGS

FIG. 1A-E: Different clinical presentation of LP in a selected set of patients.

(A) hypertrophic LP (NELP #3); (B) eruptive palmoplantar LP (NELP #7); (C) lichen planopilaris NE(LP #6); (D) non erosive oral LP (NELP #10); (E) non erosive oral LP (NELP #8).

FIG. 2A-B: Peripheral CD3⁺CD8⁺ T-cells in non-erosive LP and LSA patients express more granzyme B, perforin and CD107a than CD3⁺CD8⁺ T-cells from healthy donors.

(A) Flow cytometry detection of granzyme B, perforin and CD107a in CTL (CD3⁺CD8⁺ cells) from the blood of a patient with NELP. (B) Frequency of CTL in blood and of granzyme B, perforin and CD107a in CTL from the blood of 10 LP (NELP), 10 LSA and 7 HD. (* p<0.05; ** p<0.01; ns, non significant)

FIG. 3A-B: TCRVβ3⁺ and TCRVβ6⁺ oligoclonal expansions are skewing the peripheral blood CD8⁺ T-cell repertoire from respectively NELP and LSA patients.

(A) TCRVβ and TCRVβ6 usages in CD4⁺ and CD8⁺ T-cells from the blood of the 10 LP (NELP) patients, 10 LSA patients and in CD4⁺ and CD8⁺ T-cells from 20 sex- and age-matched HD (B) CDR3β length distributions for CD4⁺ and CD8⁺ Vβ3-Cβ rearrangements from one representative NELP patient (NELP #2) and for CD4⁺ and CD8⁺ T-cells from one representative HD. The peak of the 8^{th} codon is marked on the abscissa axis. CDR3β length distributions for CD4⁺ and CD8⁺ Vβ6-CP rearrangements from one representative LSA patient (LSA #1) and for CD4⁺ and CD8⁺ T-cells from one representative HD. (* p<0.05, ** p<0.01)

FIG. 4A-B: V-Jβ Oligoclonal expansions of CD8+ T-cells in the different patients.

CDR3β length distributions for CD8+ T-cells from LP (A) and LSA (B) patients. The Vβ-Jβ rearrangement for each patient is indicated. The peak of the codon of the oligoclonal expansion is marked on the abscissa axis.

FIG. 5A-F: A subpopulation of peripheral blood CD8⁺ T-cells from LP patients (NELP) is HPV16-specific, enriched with TCRVβ3 clonotypes and detected in lesional skin.

(A) Antibodies against capsid L1 and oncoprotein E6 were monitored by ELISA in the sera of the studied patients. (B) Flow cytometry detection using dextramer of HIV- and HPV16-specific CD8⁺ T-cells in the blood of one representative HLA-A*0201⁺ NELP patient (NELP #10). (C) Quantification of HPV-specific CD8+ T-cells in the blood of 4 HLA-A*0201 LSA patients and 3 HLA-A*0201 LP (NELP) patients. (D) Clonotype distribution estimated by CDR3β sequencing for unsorted blood T-cells or isolated blood T-cells with HPV16-dextramer for 2 different representative HLA-A*0201⁺ NELP patients (NELP #10 and NELP #5). (E) Skin in situ immunostainings using either dextramer HIV-1 (left) or dextramer HPV (right) (×1000 magnification, scale bars=10 μm) of one representative HLA-A*0201⁺ NELP patient (NELP #1). (F) CDR3β length distribution and clonotype distribution estimated by sequencing in skin lesions in NELP #3.

FIG. 6A-B: Peripheral Vβ3+ CTL in some NELP patients express granzyme, perforin and CD107a.
(a) Flow cytometry detection of granzyme, perforin and CD107a in CTL (CD3+CD8+ Vβ3+ cells)
from the blood of one patient with NELP (NELP #8).
(b) Frequency of granzyme, perforin and CD107a among Vβ3+ CTL from the blood of 9 NELP.

FIG. 7A-B: EBV-specific CTL in LSA are not enriched with TCRVβ6 clonotypes.
(a) Flow cytometry detection using dextramer of HIV- and EBV-specific CD8+ T cells in T cells from the blood of one representative HLA-A*0201+ LSA patient (LSA #7).
(b) Clonotype distribution estimated by CDR3β sequencing for unsorted T cells or isolated T cells with EBV-dextramer for 2 different representative HLA-A*0201+ LSA patients (LSA #1 and LSA #7).

### DETAILED DESCRIPTION OF THE INVENTION

To assess whether HPV16 is implicated in non-erosive forms of LP other than its erosive oral form, patients with LP, excluding patients with erosive oral LP, and patients with LSA, another cutaneous and mucosal dermatosis, were investigated.

The data obtained by the inventors demonstrate that patients with LP presenting with a wide variety of clinical manifestations display TCRVβ3⁺ CD8⁺ T-cells oligoclonal expansions in both peripheral blood and lesions, which exhibit the phenotype of CTL and recognize the HPV16 E7₁₃₋₂₄ epitope, pointing to a specific hallmark common to all clinical forms of LP, either erosive or non-erosive, and oral or non-oral. These findings extend the inventors' previous observations showing peripheral and lesional clonotypic expansions of HPV16-specific TCRVβ3⁺ CD8⁺ T-cells in patients with erosive oral LP (Viguier et al., 2015). In this previous study, the pathogenic relevance of these clonotypic expansions was supported by their consistent presence in all studied erosive oral LP patients and by their decrease in peripheral blood from several patients entering partial or complete remission under extracorporeal photopheresis (ECP), becoming even undetectable in some patients (Viguier et al., 2015). This previous study however only concerned erosive oral LP patients, and comparison with healthy patients.

These observations raise the HPV16 infection status in LP patients. Detection of viral DNA mainly derived from HPV6/11/16/31/33 has been reported in mucosal oral and/or genital lesions from LP patients in several studies (Lodi et al., 2005; Mattila et al., 2012) including the inventors' previous study in erosive oral LP (Viguier et al., 2015), providing a rationale for their role as putative antigenic stimuli. Almost all (90%) of the studied LP patients have been infected by HPV16, as shown by the detection of capsid L1-specific IgG and of oncoprotein E6-specific IgG. A causal role for HPV infection in erosive oral LP and other forms of LP does not necessarily require constant tissue viral replication over time, as an initial viral stimulation might be sufficient to trigger the founder immune response, with following autoimmune T-cell expansions related to molecular mimicry, unsequestration of masked self-epitopes or both (Selmi et al., 2012). In line with this hypothesis, molecular mimicry between HPV16 E7 protein and human self has been revealed by computer-based analyses, providing a rational basis for further investigations of LP lesional CTL regarding their immunoreactivity towards HPV16 versus self-candidate antigens (Natale et al., 2000).

These findings have several clinical and therapeutical implications for patients affected with inflammatory cutaneomucosal diseases. First, when clinical and histological features are not sufficient to confirm LP diagnosis, notably in erosive oral lesions or in some nail and scalp localizations or in non-erosive forms, complementary immunological markers such as expansions in the blood of TCRVβ3 CD8⁺ T-cells or detection of HPV16-specific T-cells in lesions would help assessing LP diagnosis. Second, the findings of the present invention, as well as those the inventors reported in erosive oral LP (Viguier et al., 2015), pave the way for innovative prophylactic and therapeutic strategies in LP. On an epidemiological perspective, the influence of the introduction of HPV16 vaccination, in particular in countries with high vaccination coverage such as Australia, is expected to reduce the incidence of LP. In terms of therapeutics, the use of antiviral treatments with an HPV-specific effect, such as cidofovir (Andrei et al., 2015), would have a limited impact on the course of LP, since complete clearance of HPV is not expected and a late therapeutical intervention on HPV has few chances to reverse the autoimmune process. On the other side, deletion or anergization of LP pathogenic T-cells, induced through immunotargeting of the CD8⁺TCRVβ3⁺ population, is expected to have a therapeutic impact in LP, and the development of anti-TCRVβ3 monoclonal antibodies as well as of bispecific CD8/Vβ3 immunoreactants could be of interest for treatment of refractory erosive oral LP, frequently requiring long-term association of immunosuppressive drugs.

Overall, while patients with LP exhibit a wide variety of clinical manifestations, clonal CD8⁺ T-cells in both peripheral blood and skin lesions from patients pinpoint a specific hallmark common to different clinical forms of LP useful in clinical settings.

The invention encompasses compositions and methods for the diagnosis and treatment of LP patients and the use of compounds to treat LP. The diagnosis can be made by detecting the presence of an immune response against an HPV infection, especially the CD8+Vβ3+ signature already mentioned. The diagnosis can additionally be made by detecting the presence of a human papilloma virus. The invention further encompasses methods for detecting a reduction in an LP symptom and methods for monitoring treatment of an LP patient.

The invention encompasses compositions and methods for the diagnosis of an HPV infection in an LP patient comprising detecting a T cell response against an HPV infection. The methods can further comprise detecting the presence of a human papilloma virus nucleic acid using a probe or primer.

### Methods for Diagnosing a Lichen Planus Patient

The invention encompasses methods for the diagnosis of LP patients, preferably non-erosive LP patients, most preferably one with a non-oral lichen planus. Based on the discovery that a massive clonal expansion of activated CD8+ T-cells with increased frequency of HPV 16-specific CD8+ T-cells is a characteristic of LP, LP patients, preferably non-erosive LP patients, can be diagnosed by providing a cell sample from a lichen planus patient. In a preferred embodiment, the method comprises providing a biological or cell sample from a lichen planus patient and detecting an immune response against a human papilloma virus infection in the sample. In another embodiment, the method comprises providing a biological or cell sample from a lichen planus patient and detecting the presence of a human papilloma virus in the sample.

Preferably, the clonal expansion of activated CD8+ T-cells, preferably of CD8+TCRVβ3+ T cells, is measured by routine techniques in the art. For example, the techniques described in the examples can be used. The clonal expansion of CD8+ TCRVβ3+ T cells refers to a frequency of CD8+ TCRVβ3+ T cells in total CD8+ T cells in a cell sample of at least 11%, preferably about 15%.

The invention is also concerned with a method for diagnosing in a patient an LP, especially an LP clinical form other than the erosive oral form of LP, preferably a NELP, comprising detecting TCRVβ3+ CD8+ T-cells oligoclonal expansions in a sample of the peripheral blood or of the lesion of the patient, suspected to have an LP. Said CD8+ T-cells preferably exhibit a cytotoxic phenotype. The oligoclonal expansions can be detected by the curve of the CDR3beta length distributions of CD8+ Vβ3-Cβ rearrangement, namely by the loss of the Gaussian-type curve, where the Gaussian-type curve is expected in the absence of oligoclonal expansion.

Preferably, the diagnosis is made on a biological sample from the patient, especially on a sample from blood, skin, scalp, nail, genital and/or mucosal lesions of said patient to be diagnosed. Such a sample preferably does not comprise HPV16 DNA.

According to another aspect, the invention is directed to a method for diagnosing an LP in a patient, especially an LP clinical form other than the erosive oral form of LP, preferably a NELP, comprising detecting a predominant usage of the TCRVβ3 gene segment in a sample of circulating CD8+ T cells of the patient with respect to the same usage in circulating CD4+ T-cells counterparts. A predominant usage corresponds to a difference between usage in CD8+ and CD4+ cells which is significant from a statistical point of view. The inventors have indeed demonstrated that LP patients, including LP patient with the non-erosive LP, display CD8+ T-cells with a skewed TCRβ3 CTL repertoire.

The invention also concerns a method for diagnosing in a patient an LP, especially an LP clinical form other than the erosive oral form of LP, preferably a NELP, comprising detecting CD8+ T cells specific for HPV16, especially oligoclonal CD8+ T cells specific for HPV16, in a blood sample of the patient, and/or in sample from lesions of the patient to be diagnosed, suspected to be LP lesions. Preferably, the sample from blood, skin, scalp, nail, genital and/or mucosal lesions of said patient to be diagnosed does not comprise HPV16 DNA. In a preferred embodiment, the CD8+ T cells specific for HPV16 are CD8+ Vβ3+ T cells. The diagnosis method thus comprises detecting CD8+ Vβ3+ T cells specific for HPV16, in a blood sample of the patient, and/or in sample from LP lesions, preferably not comprising HPV16 DNA.

Preferably, such a detection is made by flow cytometry analysis.

According to an embodiment of the preceding methods, these methods allow to discriminate between an LP and another cutaneous or mucosal dermatosis, which is not an LP, especially to discriminate between LP, e.g. NELP, and another cutaneous or mucosal dermatosis such as LSA, lichenoid GVHD, mucosal pemphigoid, psoriasis, and Brocq's pseudo alopecia. The diagnosis methods according to the invention thus provide a differential diagnosis, specifically distinguishing between an LP, including a NELP, and another cutaneous or mucosal dermatosis. The diagnosis methods of the invention thus allow to confirm the presence of an LP rather than another cutaneous or mucosal dermatosis, or, on the contrary, to confirm the absence of an LP, especially of a NELP. The claimed diagnostic methods may thus comprise a further step of concluding at the specific presence of an LP, and excluding the presence of another cutaneous or mucosal dermatosis.

In all the methods mentioned above, and in the following, a CD8+ T-cell is to be understood as a CD8+CD4− T− cell. Similarly, the reference to a CD4+ T-cell is to be understood as a CD4+CD8− T cell.

All these methods are particularly useful, when clinical and histological features are not sufficient to confirm LP diagnosis, notably in erosive oral lesion and in some nail and scalp localization. Not only these methods are very helpful for a rapid diagnostic which avoid the need for a biopsy, and the associated pain of such an intrusive act; but they are moreover more rapid, and thus allow to rapidly define the best suited treatment, depending on whether LP, especially NELP, or another type of cutaneous or mucosal dermatosis is diagnosed. Early diagnosis is indeed very important.

### Cell Sample

The cell sample can be any cell sample obtained from an LP patient. Preferably, the cell sample is generated by drawing blood, with a cytobrush, or by taking a biopsy. The cell sample is preferably a blood sample, most preferably isolated peripheral blood mononuclear cells (PBMCs), or isolated T cells. The sample can be a lymph or synovial fluid. The PBMC and T cells can be isolated by routine techniques in the art. For example, the techniques described in the examples can be used.

In some embodiments, the cell sample is a non-mucosal or non-oral cell sample. Most preferably, the non-mucosal or non-oral cell sample is a skin sample, a nail sample, or a scalp sample, or a genital sample.

The cell sample can be a tissue sample, preferably, a lesion from an LP patient. The tissue sample can be a paraffin section or thin section of a lesion.

### Immune Response Against HPV Infection

The invention encompasses detecting an immune response against an HPV infection. In one embodiment, the method comprises providing a cell sample from an LP patient, contacting the sample with an HPV peptide, and detecting the interaction between the HPV peptide and cells in the sample. Contacting the HPV peptide with a cell sample and detecting its interaction can be performed by routine techniques in the art. For example, the techniques described in the examples and below can be used.

The method can further comprise treating the patient with an anti-HPV treatment, preferably, cidofovir, and subsequently providing a second cell sample from the LP patient and repeating the step of detecting an immune response against an HPV infection.

Preferably, the HPV peptide comprises at least one HPV E6 or E7 peptide recognized by T cells, particularly CD8+ T cells. In a preferred embodiment, the HPV peptide comprises one or several peptides comprising any one of the following amino acid sequences:

(SEQ ID NO: 25)
HYNIVTFCC,
(SEQ ID NO: 26)
KLCLRFLSK,
(SEQ ID NO: 27)
KPTLKEYVL,
(SEQ ID NO: 28)
LLMGTLGIVC,
(SEQ ID NO: 29)
MLDLQPETT,
(SEQ ID NO: 30)
NTLEQTVKK,
(SEQ ID NO: 31)
RAHYNIVTF,
(SEQ ID NO: 32)
VPTLQDVVL,
and
(SEQ ID NO: 33)
YMLDLQPETT.

The method can comprise treating an LP patient with a steroid treatment, immunosuppressant treatment, or photochemotherapy, providing a cell sample from the LP patient and detecting an immune response against an HPV infection.

### MHC Multimer Staining Assay

The method can comprise an MHC multimer staining assay, for example as in Zentz et al., Human Immunology 68, 75-85 (2007) and Hoffmann et al., Int. J. Cancer: 118, 1984-1991 (2006). Since HPV-tetramer+ T cells remain many months or even years after viral clearance, multimer staining may represent a more sensitive method of HPV infection than detection of the virus itself. Wang et al., Clinical and Vaccine Immunology, Vol. 15, No. 6, June 2008, p. 937-945. Each of these references are incorporated by reference with respect to the multimer staining assays disclosed therein. MHC multimer staining assays can be performed by routine techniques in the art. For example, the techniques described in the examples and in the cited references.

T cells carry T-cell receptors (TCRs) that recognize specific MHC-peptide complexes displayed on the surface of antigen presenting cells. This specific interaction between T cells and MHC-peptide complexes has been used to detect and isolate distinct populations of T cells with specificity for a given MHC-peptide complex. MHC multimers are reagents that carry multiple MHC-peptide complexes, and thus have the ability to bind simultaneously to multiple TCRs on a single T cell, allowing for a stable interaction between the reagent and the T cell. MHC multimers can be used to detect and quantify antigen-specific T cells in fluid samples (e.g. blood, cultured cell lines, CSF, lymph, synovial fluid) by flow cytometry, and can be used for in situ detection (e.g. in solid tumors) using immunohistochemistry (IHC). MHC multimers may also be used to isolate antigen-specific T-cell populations.

MHC Dextramer® reagents (Immudex), or other similar MHC multimers, can be used. These are fluorescent labeled MHC multimers that can be used to detect antigen-specific T cells in fluid cell samples and solid tissue samples. The MHC Dextramer™ reagents come with any of three different fluorochromes (PE, APC or FITC). When using flow cytometry, they may be used to accurately monitor CD8+ T-cell responses in blood, CSF or other fluid cell samples.

In one embodiment, a MHC multimer labeled HPV peptide is contacted with a thin section of a tissue sample, preferably, a lesion from an LP patient, most preferably a non-erosive lesion, most preferably a skin lesion. The HPV peptide can bind to a specific TCR on the T cells in the sample. After contacting the HPV peptide with the cell sample, the unbound is removed and T cells that have bound the HPV peptide in the cell sample are detected. The method can be performed by routine techniques in the art. For example, the techniques described in the examples can be used. In one embodiment, cryopreserved sections are dried, fixed in acetone, incubated with TRITC-labeled anti-CD8 antibody followed by incubation with FITC-labeled Dextramer, and nuclei counter-stained with DAPI. A preferred HPV peptide is YMLDLQPETT (SEQ ID NO:33).

### Whole Blood Staining Procedure for MHC Multimers

Human peripheral whole blood can be stained with MHC Multimers, particularly MHC Dextramers™ simultaneously with immuno-phenotyping of relevant antigens, using the following protocol: Transfer 100 μL whole blood to a 12×75 mm polystyrene test tube. Add 10 μl of MHC Dextramer™ and mix with a vortex mixer. Incubate in the dark at room temperature for 10 minutes. Add an optimally titrated amount of anti-CD8 antibody (e.g. Dako clone DK25) conjugated with relevant fluorochromes and mix well. Continue incubation at 2-8° C. in the dark for 20 minutes. The staining procedure describes the use of CD8 antibody together with MHC Dextramers™. Additional antibodies for detection of extracellular antigens can be added. Add 2 mL EasyLyse™ working solution (Dako code S2364) and incubate for 10 minutes. Add 2 mL 0.01 mol/L PBS and centrifuge for 5 minutes at 300×g and aspirate supernatant.

Re-suspend pellet in an appropriate fluid for flow cytometry, e.g. 0.4 mL PBS, and analyze on a flow cytometer or store at 2-8° C. in the dark until analysis. Do not store longer than 2 hours before analysis. A preferred HPV peptide is YMLDLQPETT (SEQ ID NO:33). The assay can also be used with other cell types mentioned herein.

### TCR Assays

In one embodiment, the method comprises providing a blood sample from a lichen planus patient, preferably a NELP patient, most preferably one with a non-oral lichen planus, isolating T cells from the blood sample, and detecting CDR3β distribution of the TCRVβ3 gene segment in the T cells. In a preferred embodiment, the oligoclonal spectratyping pattern (See, e.g., Nilges et al., Journal of Virology, May 2003, p. 5464-5474 Vol. 77, No. 9, incorporated by reference) of Vβ3 TCR repertoire of sorted CD8 T cells is detected using cDNA after amplification with Vβ3 and CR specific primers.

In one embodiment, RNA from cells is extracted and reverse-transcribed into cDNA. Quantitative PCR amplifications are then done and spectratyping analysis of each Vβ-Cβ rearrangement performed. A Vβ gene segment, and more particularly Vβ3, can be used to determine whether clonal expansion exists in the tested patient. CDR3β length distribution profile allows characterization of a polyclonal T cell population (i.e., a bell-shaped curve or Gaussian-like curve) or an oligoclonal or monoclonal T cell population (i.e., a disturbed Gaussian-like curve with one or few peaks).

Thus, the method can comprise isolating RNA from cells of an LP patient, reverse-transcribing the RNA into cDNA, amplifying the DNA, and determining whether clonal expansion of a T cell population exists. Preferably, a Vβ gene segment, more particularly Vβ3, is amplified.

The method can further comprise treating the patient with an anti-HPV treatment, preferably, cidofovir, and subsequently providing a second cell sample from the LP patient, isolating T cells from the blood sample, and detecting CDR3β distribution of the TCRVβ3 gene segment in the T cells.

The method can comprise specific expansion in the blood of TCR Vβ3+CD8+ T cells, detection by quantitative PCR using the 24 Vβ-specific primers on sorted CD8+ T cells, or by flow cytometry on total blood with CD8- and Vβ3-specific antibodies.

In various embodiments, the methods used in Lim et al., J Immunol Methods. 2002 Mar. 1; 261(1-2):177-94; Blattman et al., The Journal of Immunology Dec. 1, 2000 vol. 165 no. 11 6081-6090; and Alvarez et al., Am J Transplant. 2005 April; 5 (4 Pt 1):746-56, which are incorporated by reference herein, can be used.

In one embodiment, the method comprises providing a blood sample from an LP patient, preferably an NELP patient, most preferably one with a non-oral lichen planus, optionally isolating T cells from the blood sample, and determining the CDR3β sequence of the TCRVβ3 gene segment in the T cells.

Any sequencing method known in the art can be employed. As used herein, the term “sequencing” is used in a broad sense and refers to any technique known by the skilled person including but not limited to Sanger dideoxy termination sequencing, whole-genome sequencing, sequencing by hybridization, pyrosequencing, capillary electrophoresis, cycle sequencing, single-base extension sequencing, solid-phase sequencing, high-throughput sequencing, massively parallel signature sequencing (MPSS), sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, 454 sequencing, Solexa Genome Analyzer sequencing, SOLiD® sequencing, MS-PET sequencing, mass spectrometry, and a combination thereof. In specific embodiments, the method of the invention is adapted to run on ABI PRISM® 377 DNA Sequencer, an ABI PRISM® 310, 3100, 3100-Avant, 3730, or 3730x1 Genetic Analyzer, an ABI PRISM® 3700 DNA Analyzer, or an Applied Biosystems SOLiD™ System (all from Applied Biosystems), a Genome Sequencer 20 System (Roche Applied Science).

Preferably, the CDR3β sequence of the TCRVβ3 gene segment in the T cells of the cell sample encodes any one of the following amino acid sequences: SRTVNTI (SEQ ID NO:1), SLVETGFIGTTFEVEQ (SEQ ID NO:2), SLGVGYEQ (SEQ ID NO:3), SLRGAYEQ (SEQ ID NO:4), SFQGFETQ (SEQ ID NO:5), SLSTPNYEQ (SEQ ID NO:6), SFQGYYEQ (SEQ ID NO:7), SWTRTYNEQ (SEQ ID NO:8), SFGLRAGTYEQ (SEQ ID NO:9), SLTSATGEL (SEQ ID NO:10), SLRGAINGEL (SEQ ID NO:11), SLWAGNLREQ (SEQ ID NO:12), a consensus peptide consisting of: S in position 1, L, V or F in position 2, G or R in position 3, V or G in position 4, G, A or H in position 5, Y in position 6, E in position 7, and Q in position 8 (SEQ ID NO:60).

Preferably, the CDR3β sequence of the TCRVβ3 gene segment in the T cells of the cell sample comprises one of the following nucleic acid sequences:

(SEQ ID NO: 13)
AGTCGGACGGTAAACACCATA,
(SEQ ID NO: 14)
AGTTTGGTTGAAACAGGTTTTATAGGGACTACGTTTGAGGTTGAGCAG,
(SEQ ID NO: 15)
AGTTTGGGTGTGGGCTACGAGCAG,
(SEQ ID NO: 16)
AGTCTACGGGGGGCCTACGAGCAG,
(SEQ ID NO: 17)
AGTTTTCAGGGGTTCGAGACCCAG,
(SEQ ID NO: 18)
AGTTTATCGACTCCTAACTACGAGCAG,
(SEQ ID NO: 19)
AGTTTTCAGGGGTACTACGAGCAG,
(SEQ ID NO: 20)
AGTTGGACTAGGACTTACAATGAGCAG,
(SEQ ID NO: 21)
AGTTTTGGTCTTCGAGCGGGGACCTACGAGCAG,
(SEQ ID NO: 22)
AGTTTGACTAGCGCCACCGGGGAGCTG,
(SEQ ID NO: 23)
AGCCTCCGGGGGGCTATTAACGGGGAGCTG,
and
(SEQ ID NO: 24)
AGTTTATGGGCAGGGAATTTACGTGAGCAG

In one embodiment, the method comprises detecting any of the nucleic acid sequences of SEQ ID Nos 13-24 or any nucleic acid sequence encoding any of the amino acid sequences of SEQ ID NOs 1-12. These sequences can be specifically detected by numerous techniques known in the art, such as sequencing, hybridization, or amplification assays.

For example, the amplification method can be RCA, MDA, NASBA, TMA, SDA, LCR, b-DNA, PCR (all forms including RT-PCR), RAM, LAMP, ICAN, SPIA, QB-replicase, or Invader.

The method can further comprise treating the patient with an anti-HPV treatment, preferably, cidofovir, and subsequently providing a second blood sample from a LP patient, and determining the CDR3β sequence of the TCRVβ3 gene segment in the T cells in the blood sample.

The invention encompasses methods for detecting expansion of a clonal population of CD8+ TCRVbeta6+ T cells in a sample. In a preferred embodiment, the patient is suspected to have a lichen sclerosus et atrophicus (LSA)

In one embodiment, the method comprises detecting the presence of a clonal expansion of CD8+ T cells in a blood sample of a patient suspected to have a lichen sclerosus et atrophicus (LSA), said method comprising isolating T cells from a blood sample from the patient and detecting expansion of a clonal population of CD8+ TCRVbeta6+ T cells in the sample, wherein said clonal population has a single CDR3beta sequence in the TCRVbeta6 gene segment.

In one embodiment, the CDR3beta sequence in the TCRVbeta6 gene segment in the CD8+ T cells encodes any one of the following peptides: SSMGVTEASEQ (SEQ ID NO: 48), SFWQVPGEL (SEQ ID NO: 49), SPYRSSYNEQ (SEQ ID NO: 50), SSTSWGETQ (SEQ ID NO: 51), SLGRGPGDTQ (SEQ ID NO: 52), SHQTDEA (SEQ ID NO: 53), STRTGRFEKL (SEQ ID NO: 54), SSAGQGYTEA (SEQ ID NO: 55), SSPTGGLDEAFF (SEQ ID NO: 56), SPPTGDYEQYF (SEQ ID NO: 57), SSNTASSYNEQ (SEQ ID NO: 58), SNSTSGYGY (SEQ ID NO: 59).

The methods can be performed by routine techniques in the art. For example, the techniques described in the examples can be used.

### HPV Detection

The invention encompasses methods of detecting the presence or absence of a human papilloma virus. The method can comprise detecting the presence or absence of HPV DNA, RNA, or protein in an LP patient sample, preferably an NELP patient sample, most preferably one with a non-oral lichen planus. The method can comprise preparing nucleic acids from the cell sample and contacting the nucleic acids with an HPV specific primer or probe. The nucleic acids can be DNA and/or RNA. The method can be performed by routine techniques in the art. For example, the techniques described in the examples can be used.

HPV can also be detected by preparing a nucleic acid from cells from an LP patient and sequencing the HPV nucleic acid. Any sequencing method known in the art can be employed.

For all technologies described herein, although in some embodiments the primers are used in solution, in other embodiments the primers are linked to a solid support.

The method can further comprise treating the patient with an anti-HPV treatment, preferably, cidofovir, and subsequently providing a second cell sample from the LP patient and repeating the step of detecting the presence or absence of a human papilloma virus.

The method can comprise treating an LP patient, preferably an NELP patient, most preferably one with a non-oral lichen planus, with a steroid treatment, immunosuppressant treatment, or photochemotherapy, providing a cell sample from the LP patient and detecting the presence or absence of a human papilloma virus.

### Anti-HPV Treatment

The method can further comprise treating the patient, preferably an NELP patient, most preferably one with a non-oral lichen planus, with an anti-HPV treatment. Within the context of the invention, an anti-HPV treatment includes HPV vaccines, cytotoxic agents, immunomodulators, and antiviral compounds. Subsequently, a second cell sample can be provided from the LP patient for further determinations.

The method can comprise treating an HPV positive lichen planus patient, preferably an NELP patient, most preferably one with a non-oral lichen planus, with a suitable treatment. In some embodiments the treatment is by a method comprising administering a composition comprising at least one early and/or at least one late protein of an HPV virus, or a fragment thereof. The method can comprise treating an HPV positive lichen planus patient with a treatment comprising administering a composition comprising the major capsid protein L1 of HPV types 6, 11, 16, and 18. The method can comprise treating an HPV positive lichen planus patient with a treatment comprising administering a composition comprising the major capsid protein L1 of HPV types 16, and 18. The method can comprise treating an HPV positive lichen planus patient with a treatment comprising administering a composition comprising at least one chimeric recombinant Bordetella sp. adenylate cyclase (CyaA) protein or fragment thereof. In some embodiments the CyaA protein or fragment thereof comprises at least one inserted human papilloma virus (HPV) E6 and/or E7 epitope. Additional treatments are disclosed, for example, in Hung et al., Expert Opin Biol Ther. 2008 April; 8(4): 421-439.

The method can comprise treating an HPV positive lichen planus patient, preferably an NELP patient, most preferably one with a non-oral lichen planus, with a treatment comprising administering a composition comprising a live-vector based vaccine. For example, the live-vector based vaccine may be a bacterial vector-based vaccine, such as Salmonella typhimurium or Listeria monocytogenes, or a viral vector-based vaccine, such as adenovirus (AdV).

The method can comprise treating an HPV positive lichen planus patient, preferably an NELP patient, most preferably one with a non-oral lichen planus, with a treatment comprising administering a composition comprising a peptide and/or protein-based vaccine.

The method can comprise treating an HPV positive lichen planus patient, preferably an NELP patient, most preferably one with a non-oral lichen planus, with a treatment comprising administering a composition comprising a DNA-based vaccine.

The method can comprise treating an HPV positive lichen planus patient, preferably an NELP patient, most preferably one with a non-oral lichen planus, with a treatment comprising administering a composition comprising an RNA replicon-based vaccine.

In some embodiments the treated patient is infected with a high risk HPV virus. In some embodiments the treated patient is infected with a human papilloma virus is of a type selected from types 16, 18, 31, 33, 35, 39, 45, 51, 52, 56, 58, 59, 68, 69, 73, and 82.

The method can comprise treating the patient, preferably an NELP patient, most preferably one with a non-oral lichen planus, with an HPV vaccine, preferably a therapeutic vaccine. The vaccine can be a peptide-based vaccine, a protein-based vaccine, a live vector-based vaccine, a whole cell-based vaccine or a DNA vaccine. Barrios et al., Cancer Immunol Immunother. 2012 August, 61(8):1307-17; Lin et al., Immunol Res. 2010 July, 47(1-3): 86-112; Li et al., Oncology Reports 24: 1323-1329, 2010; Preville et al., Cancer Res 2005; 65:641-649; Zanotto et al. Journal of Translational Medicine 2011, 9:190; Hung et al., Expert Opin Biol Ther. 2008 April; 8(4): 421-439.

The method can comprise treating the patient, preferably an NELP patient, most preferably one with a non-oral lichen planus, with an immunomodulator. Immunomodulators include imiquimod and cidofovir.

The method can comprise treating the patient, preferably an NELP patient, most preferably one with a non-oral lichen planus, with an antiviral compound. Antiviral compounds include small molecule inhibitors. In one embodiment, compounds PFL #13 or #14 of Huang et al., Antiviral Res. 2012 February, 93(2): 280-287, can be used. In one embodiment, compounds #1, 2, or 3 of White et al., J. Biol. Chem. 2003, 278:26765-26772, can be used. Their structures are incorporated herein by reference.

### Methods for Detecting a Reduction in a Lichen Planus Symptom

The invention includes methods comprising a step of detecting a reduction in a LP symptom. The primary lesion of LP is a small opalescent papule, whitish and keratotic (not removable with a spatula). Nico et al., An Bras Dermatol. 2011; 86(4):633-43. The detection of a reduction in a LP symptom can be performed using standard clinical criteria.

### Methods for Monitoring Treatment of a Lichen Planus Patient

The invention includes methods for monitoring treatment of an LP patient, preferably an NELP patient, most preferably one with a non-oral lichen planus. These methods can comprise treating an LP patient, preferably an NELP patient, with an anti-HPV treatment or preferably with a steroid treatment, immunosuppressant treatment, or photochemotherapy and determining whether the treatment has decreased an HPV-induced immune response, preferably a T-cell response, against a human papilloma virus infection in a biological sample from the treated patient. Most preferably T-cell response is a CD8+ TCRVbeta3+ T cell response, most preferably T-cell response is a HPV16-specific CD8+ TCRVbeta3+ T cell response, or the T-cell response is a HPV16-specific CD8+ T cell response. The biological sample is blood, or a lesional biopsy, more particularly a lesional biopsy of skin, nail, genital organ or scalp. The method for monitoring treatment can further comprises steps of evaluating an LP symptom before and after the treatment. The decrease of the HPV-induced T cell response is determined by quantifying HPV-induced T cell response before and after the treatment. These methods can comprise treating an LP patient, preferably an NELP patient, most preferably one with a non-oral lichen planus, with an anti-HPV treatment, or preferably with a steroid treatment, immunosuppressant treatment, or photochemotherapy, and determining whether the treatment has decreased an LP symptom in the patient. In case no modification or modulation of the HPV-induced immune response is observed, it can be concluded that the treatment is without effect on the LP.

The methods for monitoring treatment of an LP patient according to the invention may comprise determining in a biological sample obtained from said patient, the usage of the TCRVβ3 gene segment in circulating CD8+ T cells, before and after treatment, and detecting a modulation of the TCRVβ3 gene segment usage, preferably a decrease, or alternatively the absence of a decrease when the treatment is inoperative or without effect on the LP. The efficacy of a LP treatment, especially a NELP treatment can thus be tested by these methods. In another embodiment, the methods for monitoring the treatment of an LP patient may comprise assaying oligoclonal CD8+ T cells specific for HPV16 in a biological sample obtained from the patient, before and after treatment, and detecting a decrease of the number of CD8+ T cells specific for HPV16, preferably CD8+Vβ3+ T cells specific for HPV16. The LP patient can be an NELP patient, most preferably one with a non-oral lichen planus, or a LP patient without an erosive oral LP. The treatments are as disclosed above and include anti-HPV treatment or preferably a steroid treatment, immunosuppressant treatment, or photochemotherapy. The biological sample is blood, or a lesional biopsy, more particularly a lesional biopsy of skin, nail, genital organ or scalp. The method for monitoring treatment can further comprises steps of evaluating an LP symptom before and after the treatment. The absence of modification of the number of CD8+ T cells specific for HPV16 indicated that the treatment is without effect.

According to an embodiment, the biological sample of said patient does not comprise HPV16 DNA.

### Methods and Compositions for the Treatment of Lichen Planus

The invention encompasses methods and compositions for treating an LP patient, preferably an NELP patient, most preferably one with a non-oral lichen planus.

In one embodiment, the method comprises specifically immunotargeting subsets of T cells. In one embodiment, the method comprises immunotargeting of the CD8+ TCRVβ3+ population in order to delete or anergise this subset. In one embodiment, a monoclonal antibody or other cell binding domain can be spliced to a toxin domain. Knechtle, Phil. Trans. R. Soc. Lond (2001) 356, 681-689.

In one embodiment, the method for treating an LP patient comprises the steps of immunotargeting the CD8+ TCRVβ3+ T-cell population of said patient, and deleting or anergizing said T-cell population, thus treating the LP. Preferably, the LP is an LP clinical form other than the erosive oral form of LP, most preferably a NELP.

In one embodiment, anti-TCRVβ3 monoclonal antibodies are used. In one embodiment, bispecific CD8/Vβ3 immunoreactants are used.

In one embodiment, the method comprises immunotargeting of the CD8+ TCRVβ3+ Jβ2.7 population in order to delete or anergise this subset. In one embodiment, the specific sequence of one of SEQ ID NOs: 1-12 is targeted in order to delete or anergise this subset. In one embodiment, a monoclonal antibody against any of SEQ ID NOs: 1-12 is be spliced to a toxin domain in order to delete or anergise this subset.

In one embodiment, the method comprises immunotargeting of the CD8+ TCRVβ3+HPV+ population. In one embodiment, an HPV-specific multimer, preferably an HPV16-specific multimer coupled to a toxin, is used to delete or anergise this subset. See, e.g., Gojanovich et al., Journal of Diabetes Science and Technology Volume 6, Issue 3, May 2012; Vincent et al., J Immunol 2010, 184:4196-4204, which are hereby incorporated by reference. In one embodiment, the HPV16-specific multimer comprises at least one amino acid sequence selected from SEQ ID NOs:21-29.

Preferably, the toxin is doxorubicin or saporin.

In one embodiment, the method comprises treating the patient, preferably an NELP patient, most preferably one with a non-oral lichen planus, with (i) an anti-HPV treatment or (ii) a steroid treatment, immunosuppressant treatment, or photochemotherapy. In one embodiment, the method comprises treating the patient with (i) an anti-HPV treatment and (ii) a steroid treatment, immunosuppressant treatment, or photochemotherapy. Preferably, the patient is treated with photochemotherapy. Preferably, the anti-HPV treatment is cidofovir.

In one embodiment, the method further comprises detecting the presence of an immune response against a human papilloma virus infection in a cell sample from the patient, preferably an NELP patient, most preferably one with a non-oral lichen planus. Any of the assays described herein or known to the skilled artisan can be used.

The invention comprises the use of any of the above compounds for the treatment of lichen planus, preferably an NELP, most preferably a non-oral LP, and the use of any of the above compounds for the preparation of a medicament for treating an LP patient, preferably an NELP patient, most preferably one with a non-oral lichen planus.

The invention also encompasses methods for treating a lichen planus patient, preferably a NELP patient, most preferably one with a non-oral lichen planus, comprising selecting an HPV positive lichen planus patient and treating the HPV positive lichen planus patient, preferably an NELP patient. As shown in the examples, one way that such patients may be treated is by a method that comprises extracorporeal photochemotherapy (ECP). Accordingly, the invention also encompasses a method for treating a lichen planus patient comprising selecting an HPV positive lichen planus patient; and treating the HPV positive lichen planus patient with extracorporeal photochemotherapy (ECP). The patient can be HPV16. In one embodiment, selecting an HPV positive lichen planus patient comprises detecting the presence of a human papilloma virus or an immune response against a human papilloma virus infection in a cell sample from the patient. Any of the assays described herein or known to the skilled artisan can be used.

The invention also encompasses methods for treating a lichen planus patient, preferably an NELP patient, most preferably one with a non-oral lichen planus, comprising selecting a lichen planus patient infected with a high-risk strain of HPV and treating the high-risk HPV positive lichen planus patient. In some embodiments the high-risk strain is selected from HPV types 16, 18, 31, 33, 35, 39, 45, 51, 52, 56, 58, 59, 68, 69, 73, and 82. In some embodiments the patient is a patient with a persistent high risk HPV infection. In some embodiments the treatment prevents lichen planus in the patient. In some embodiments the treatment ameliorates lichen planus in the patient.

The method can comprise treating an HPV positive lichen planus patient, preferably an NELP patient, most preferably one with a non-oral lichen planus, with a treatment selected from a composition comprising the major capsid protein L1 of HPV types 6, 11, 16, and 18; a composition comprising the major capsid protein L1 of HPV types 16, and 18; and at least one chimeric recombinant Bordetella sp. adenylate cyclase (CyaA) protein or fragment thereof, the CyaA protein or fragment thereof comprising at least one inserted human papilloma virus (HPV) E7 epitope. In some embodiments the human papilloma virus is a high-risk HPV virus. In some embodiments the human papilloma virus is of a type selected from types 16, 18, 31, 33, 35, 39, 45, 51, 52, 56, 58, 59, 68, 69, 73, and 82.

In a further aspect, the invention also relates to a kit or kit-of-part, for detecting or diagnosing a LP in a patient, preferably an NELP, and most preferably a non-oral LP. Such a kit allows to detect CD8+ T-cells, especially TCRVβ3+CD8+ T-cells, and preferably to quantify these cells. The kit according to the invention preferably comprises at least:
an antibody recognizing T-cells, e.g. an anti-CD3 antibody,
an antibody recognizing CD8+ T cells, e.g. an anti-CD8 antibody,
an antibody recognizing CD4+ T cells, e.g. an anti-CD4 antibody and
an antibody recognizing TCRVβ3+ T cells, e.g. an anti-TCRVβ3 antibody.

Such a kit allows to detect and quantify the TCRVβ3 usage in CD8+ T-cells, and to compare to the TCRVβ3 usage in CD4+ T-cells of a sample, and thus to diagnose a Lichen Planus to a patient, even in the absence of erosive oral LP or of oral LP symptoms. Such an LP is diagnosed if the TCRVβ3 usage in CD8+CD4− T-cells is statistically increased with respect to TCRVβ3 usage in CD4+CD8− T-cells.

The kit of the invention may also comprise a dextramer-HPV16, thus allowing to detect the Vβ3+CD8+CD4− HPV16− T-cells. Presence of such cells is indicative of LP, especially in patients having no symptoms of erosive oral LP or of erosive LP. In a preferred embodiment, this kit is to be used on a sample of a patient to be diagnosed, affected with inflammatory cutaneous or mucosal disease; even more preferably for samples without HPV16 DNA.

The invention further encompasses the embodiments set forth in the examples and combinations of them with the embodiments disclosed herein.

### EXAMPLES

### Summary

Lichen planus (LP) is a cutaneomucosal chronic inflammatory disease characterized by a CD8⁺ cytotoxic T-lymphocytes (CTL) infiltrate. In erosive oral LP, the inventors found HPV16-specific activated CTL in lesions, supporting a pathogenic contribution of HPV16. Here, they investigated whether a similar scenario occurs in other clinical forms of LP and in lichen sclerosus et atrophicus (LSA), another chronic disease also affecting the mucosa and/or the skin. Blood CTL from LP and LSA patients expressed significant higher levels of granzyme B, perforin and CD107a proteins than healthy donors. Expansions of TCRVβ3⁺ CTL, with presence of TCR clonotypes identical to those detected in erosive LP, were found both in blood and mucosal/skin lesions of LP, but not of LSA patients. These expansions were enriched with HPV16-specific CD8⁺ T-cells as shown by their recognition of the E7₁₃₋₂₄ immunodominant epitope. In LSA patients, the peripheral repertoire of CTL was oligoclonal for TCRVβ6⁺ CTL, and those clonotypes did not show HPV16-specificity. Finally, although patients with LP and LSA have developed antibodies against HPV16 capsid L1, antibodies against HPV16 E6 were only observed in patients with LP. Overall, the data collectively support a causal involvement of HPV16-specific CTL in the different clinical forms of LP, not only in erosive oral LP, while a different scenario operates in LSA.

### Example 1. Patients and Healthy Donors

Ten patients with active LP, excluding patients with erosive oral LP, and 10 with active LSA were enrolled. The Institutional Review Board (Comité de Protection des Personnes Ile de France IV, n^{o} IRB 00003835) approved the study (EUDRACT n^{o} 2015-A01697-42). The patients were fully informed and gave a written consent for blood and skin collections. For all of them, blood and cutaneous samples of lesions for HPV detection (see below) were collected. Skin lesion biopsies for research purpose were optional and realized in a subset of willing patients as described in Table 1. Healthy donors (HD) were blood donors from the French National Blood Service (EFS), 7 were analyzed in flow cytometry and 20 sex and age-matched to the 10 LP and 10 LSA patients were analyzed in molecular biology studies. The study was approved by the French South-West & Overseas ethical committee and was registered at the French Ministry of Higher Education and Research (DC-2015-2488). All experiments were performed in agreement with the guidelines of the Declaration of Helsinki.

### Example 2. Antibodies and Flow Cytometry Analysis

Fresh blood cells were stained with anti-CD3-PerCP-Cy™ 5.5 (Clone UCHT1, BD Biosciences), anti-CD45-APC-H7 (2D1, BD Biosciences), anti-CD8-V500 (SKi, BD Biosciences), Dextramer-HPV16 E713-24-PE (Dext-HPV PE; HLA-A*0201; YMLDLQPETT, Immudex), Dextramer-HIV-1 P17 Gag 77-85-PE (Dext-HIV-PE; HLA-A*0201; SLYNTVATL, Immudex), anti-TCR-Vβ3-FITC (JOVI-3, Ancell), anti-HLA-A2 PE-Cy7 (BB7.2, BD Biosciences), anti-Perforin PE-CF594 (δG9, BD Biosciences), anti-Granzyme B BV421 (GB 11, BD Biosciences), anti-CD107a-APC (H4A3, BD Biosciences) were used. Cells were incubated with dextramer for 10 min at room temperature, then antibodies were added for 30 min at 4° C. Stained cells were analyzed using a LSR Fortessa flow cytometer (BD Biosciences).

### Example 3. Purification of T-Lymphocyte Subsets

Dead cells were removed using the MACS® Dead Cell Removal kit. T-cells were isolated using anti-CD8 or anti-CD4 Ab-coated immunomagnetic beads (Miltenyi Biotec). Cell purities exceeded 95%. CD3⁺CD8⁺TCRVβ3⁺Dext-HPV16⁺ or Dext-HPV16⁻ populations were isolated using a MoFlo® Astrios™ cytometer cell sorter (Beckman Coulter).

### Example 4. T-Cell Receptor Repertoire Analysis

RNA was extracted using RNAeasy kit (Qiagen) and reverse-transcribed into cDNA using Superscript II (Invitrogen Life Technologies). Quantitative PCR amplifications were performed with TaqMan Universal PCR Master Mix (Applied Biosystems), TCRVβ gene segments specific oligonucleotides, TCRCβ-specific antisense primer and a fluorescent probe specific for the TCRCβ gene segment (Fazilleau et al., 2007).

### Example 5. Cloning and Sequencing of CDR3β Rearrangements

Vβ-Jβ PCR products were cloned in pCR® 4Blunt TOPO vector (Invitrogen Life Technologies). Sequencing reactions were performed using the ABI PRISM Big Dye Terminator Reaction Kit (Applied Biosystem). Reaction products were analyzed on an ABI 3130XL 16 capillaries (Applied Biosystems).

### Example 6. Detection of Human Papillomavirus Infection

Cutaneous samples were obtained from lesions using a cytobrush (DNAPAP Cervical Sampler; Qiagen). The brush was then placed in 1 mL of Cervical Specimen Transport Medium (Digene). DNA was extracted with the QIAamp DNA minikit (Qiagen) according to the manufacturer's instructions. The presence of alpha, beta and gamma HPV DNA was detected by a type-specific multiplex genotyping assay, which combines multiplex PCR and hybridization to type-specific oligonucleotide probes on fluorescent beads (Luminex corporation), as described previously (Gheit et al., 2006; Schmitt et al., 2010; Clifford et al., 2016). After PCR amplification, each reaction mixture was analyzed by multiplex HPV genotyping using Luminex technology (Luminex corporation) as described previously (Schmitt et al., 2010). Search for serum IgG antibodies against HPV16 major capsid protein L1 was performed using ELISA (Combita et al., 2002). Serum samples were tested and virus like particle- or E6 bound antibodies were detected using anti-human IgG conjugated to peroxidase (Southern Biotech). For detection of anti-HPV16 E6 antibodies, an Escherichia coli-expressed fusion protein composed of HPV16 E6 fused at the C-terminus of glutathione-S-transferase (GST-E6) was generated and coated in microplate wells. For both assays, cut-off value for positivity based on samples from young children (mean reactivity plus 3 SD) was 0.2.

### Example 6. In Situ Immunostaining

Acetone-fixed cryosections were incubated with dextramer for 75 minutes at room temperature and mounted with VECTASHIELD® Mouting Medium with DAPI (Vector Laboratories). Slides were studied using an Axiovert 200M microscope with MRm camera (Zeiss).

### Example 7. Statistical Analysis

Data are presented as means±standard error of the mean. Prism software (Graph Pad) was used for statistical analysis. Statistical analysis was performed by unpaired Mann-Whitney test. A p value of less than 0.05 was considered significant for all analyses.

### Example 8. Circulating CD8+ T-Cells of NELP and LSA Patients Display a Cytotoxic Phenotype

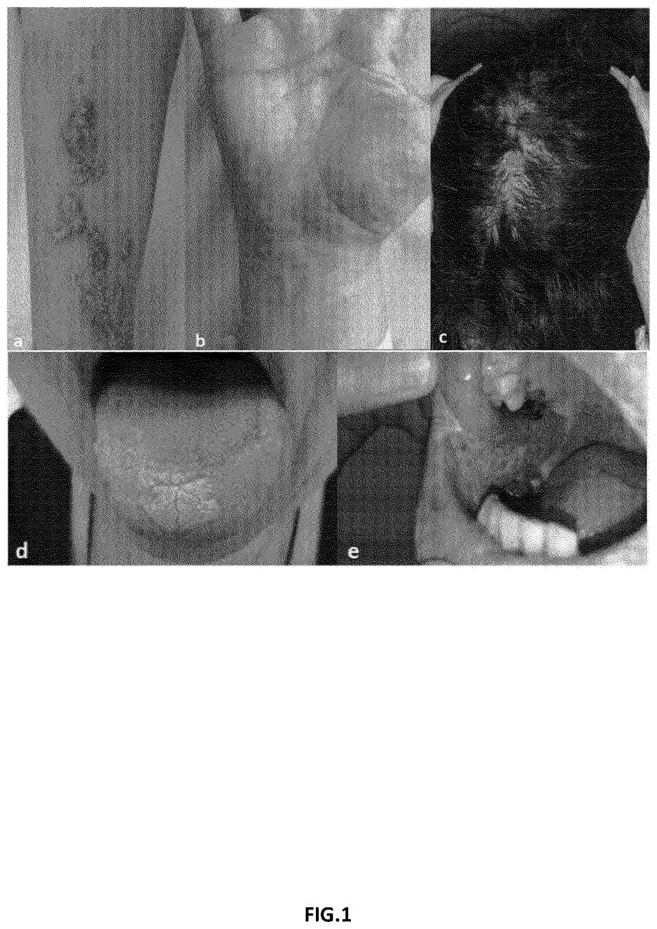

To assess whether HPV16 is implicated in other form of LP than its erosive oral form, we investigated patients with LP, excluding patients with erosive oral LP, and patients with LSA, another cutaneous and mucosal dermatosis. Briefly, 10 patients diagnosed with LP, with a mean age of 50.8 years and a mean duration of lichen of 12 years, presenting with active lesions of papular LP, bullous LP, hypertrophic LP, non-erosive oral LP and/or lichen planopilaris were included (Table 1 and FIG. 1). The 10 LSA patients had a mean age of 58.1 years (range: 28-76) and had active LSA affecting the vulva or the glans, evolving for a mean duration of 7.4 years and treated with topical steroids (Table 2). All the studied patients had negative serologies against HIV, HCV or HBV infection.

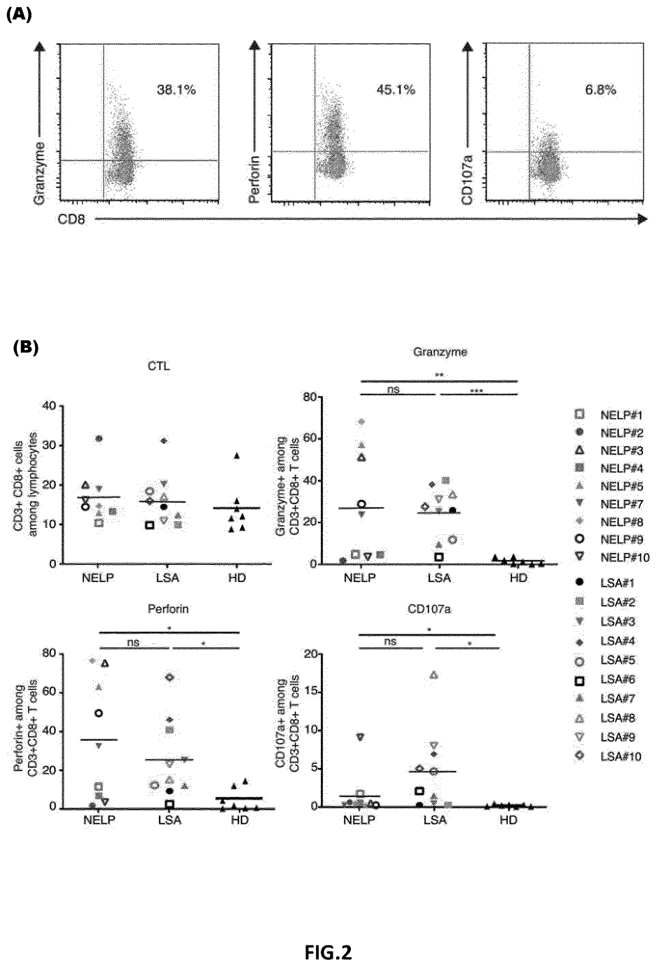

First, we analysed the phenotype of peripheral blood CD3⁺CD8⁺ T-cells from these patients using flow cytometry, and we monitored granzyme B, perforin and CD107a contents among these cells (FIG. 2A). We found that the proportions of circulating CD3⁺CD8⁺ T-cells among total lymphocytes were similar in both LP and LSA patients, and in line with rates observed in healthy donors (HDs) (FIG. 2B, top left panel). In contrast, the mean frequencies of CD3⁺CD8⁺ T-cells expressing intracellular granzyme B, perforin or surface CD107a were higher in LP and LSA patients as compared to HDs (FIG. 2B, granzyme B: 24.9±8.1/24.2±4.0/1.1±0.5; perforin: 31.9±9.8/24.3±6.5/4.8±2.2; CD107a: 1.8±0.9/4.5±1.7/0.2±0.1 for LP, LSA and HD respectively). Of note, no significant differences were observed between LP and LSA patients for all the parameters tested (FIG. 2). Overall, we observed that peripheral CD3⁺CD8⁺ T-cells from LP and LSA patients exhibit a cytotoxic phenotype.

### Example 9. Peripheral Blood CD8⁺ T-Cell Repertoire from LP and LSA Patients are Characterized by TCRVβ3⁺ and TCRVβ6⁺ Oligoclonal Expansions Respectively

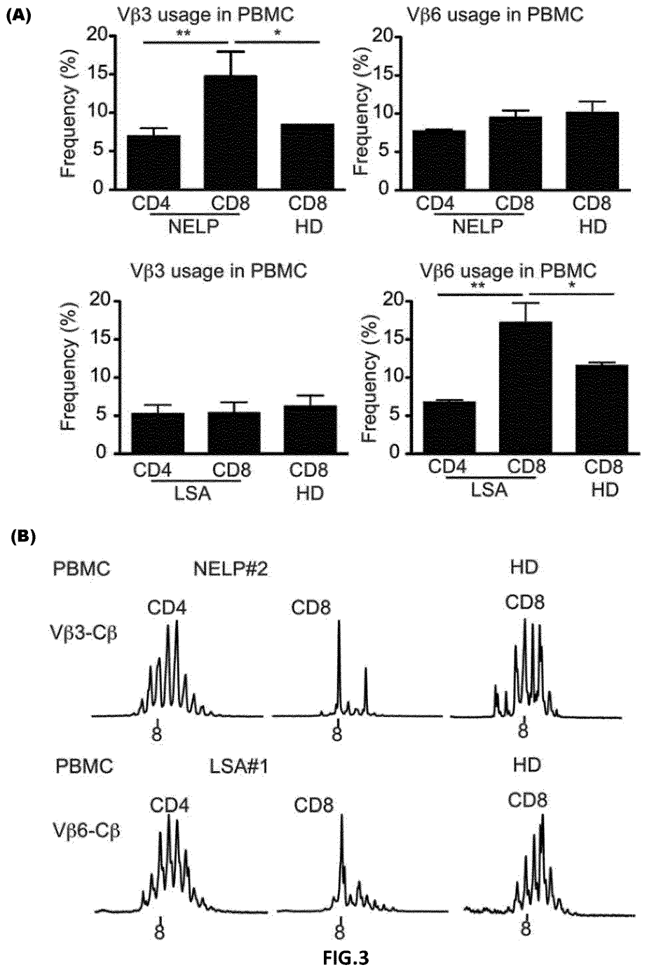

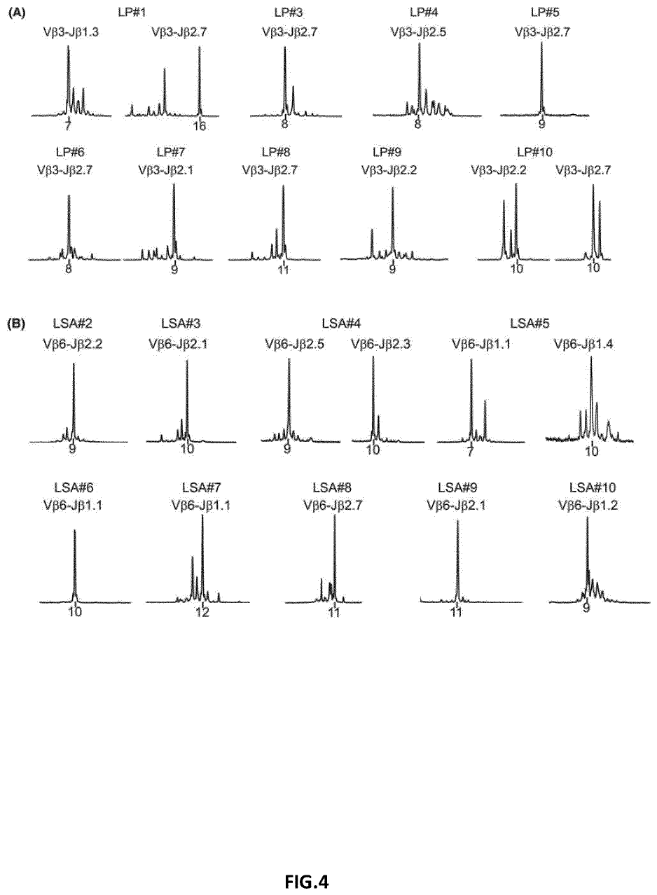

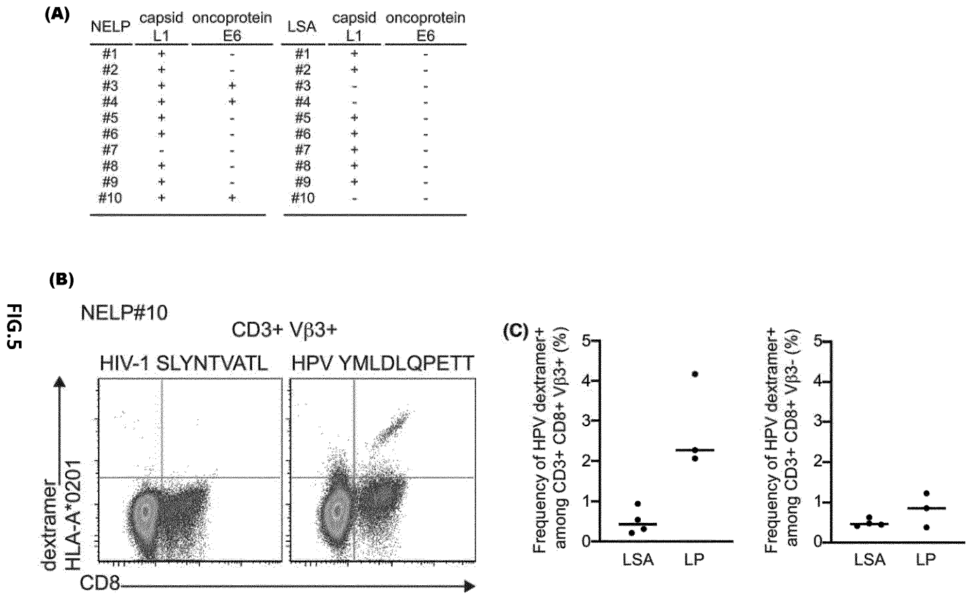

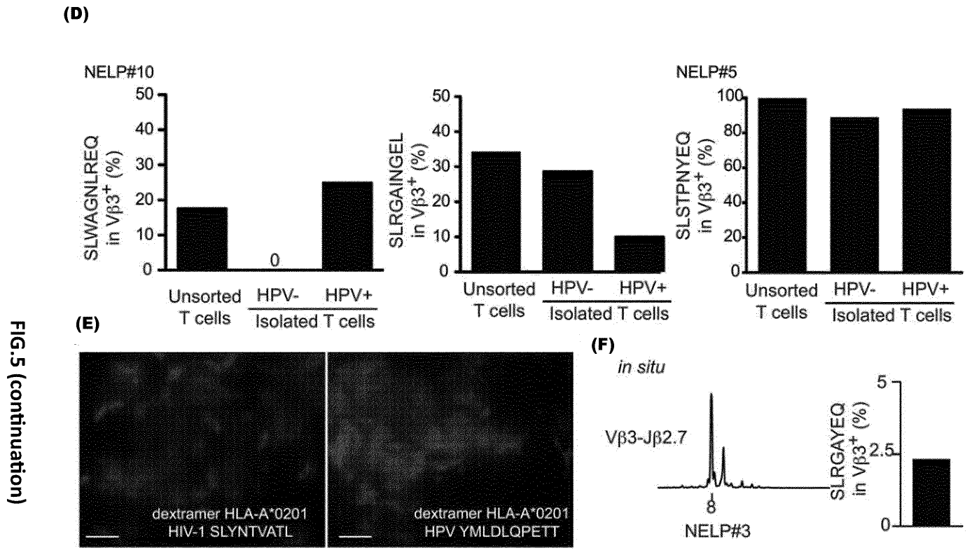

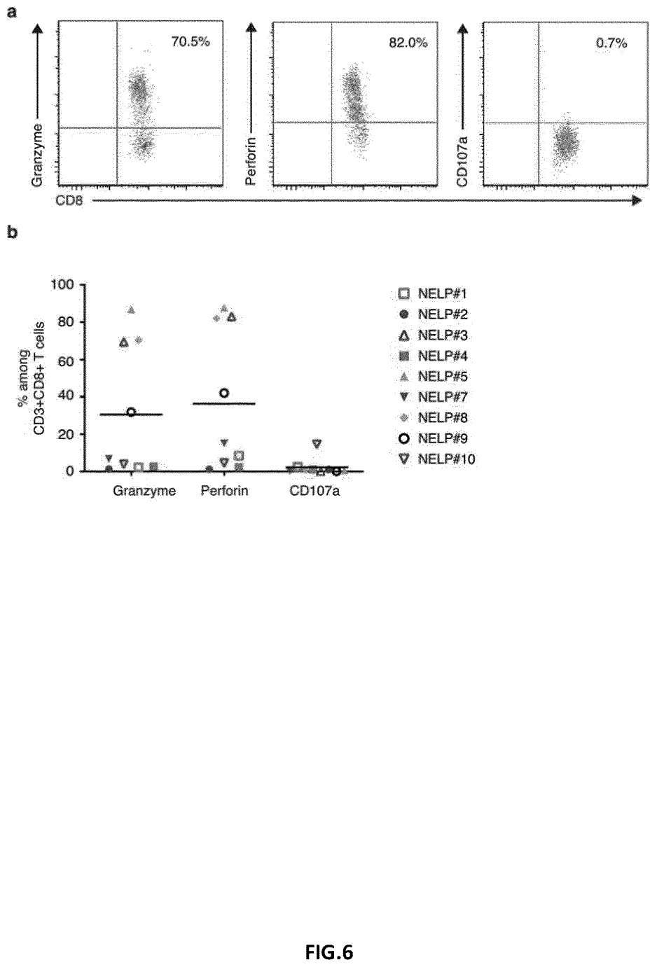

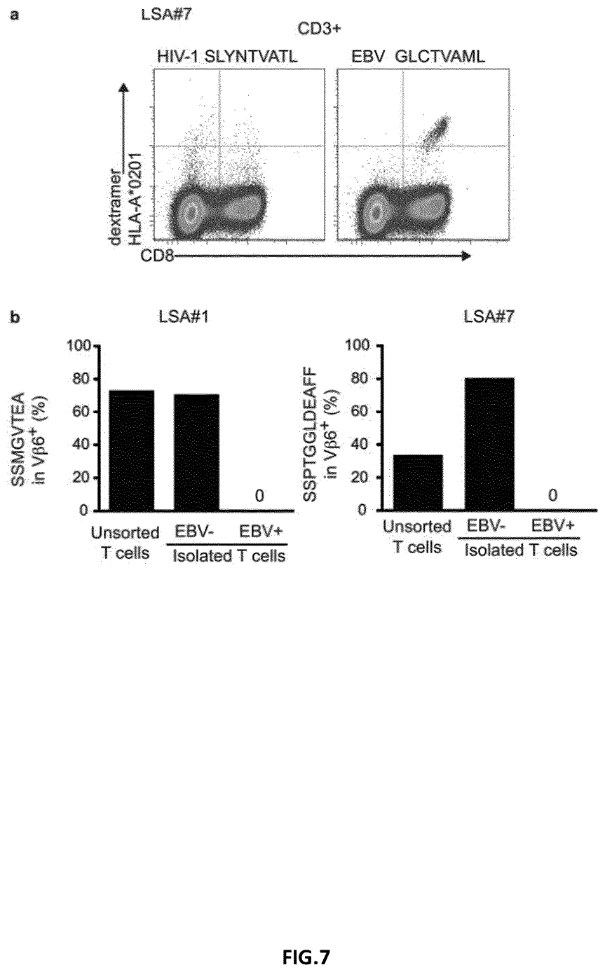

Since we previously observed CTL oligoclonal expansions in patients with erosive oral LP lesions, we assessed TCRVβ gene segment usage by RT-qPCR in sorted CD4⁺ and CD8⁺ blood T-cells from the 10 NELP, 10 LSA and CD4⁺ and CD8⁺ T-cells from 20 HD. All TCRVβ gene segments were amplified in all studied cell populations. Interestingly, as observed for patients with erosive oral LP, we detected a predominant usage of the TCRVβ gene segment in circulating CD8⁺ T-cells of LP patients (14.76%±3.18), while this gene segment was used at a much lower level in circulating CD4⁺ T-cell counterparts from the same patients (6.98%±1.02), and in CD4⁺ and CD8⁺ blood T-cells from sex- and age-matched HD (7.52%±1.04 and 8.47%±1.90 respectively) (FIG. 3A). Interestingly, expression of cytotoxic molecules among Vb3+ CTL also displayed a bimodal expression among the tested patients, with 4 out of 9 that had high levels (NELP #3, NELP #5, NELP #8, NELP #9) (See FIG. 6). Regarding patients with LSA, they did not display skewed TCRVβ gene segment usage, since it was used at 5.07%±0.79, 7.37%±2.37, 8.04%±1.83 and 8.36%±2.03 in CD4⁺ and CD8⁺ T-cells from LSA patients and in CD4⁺CD8⁺ T-cells from matched HD, respectively (FIG. 3A). In contrast, TCRVβ6 gene segment usage was largely increased in peripheral CD8⁺ T-cells from LSA patients (15.24%±1.9), while it was detected at similar levels in circulating CD4⁺ counterparts from the same patients (7.24%±0.26), in CD4⁺ and CD8⁺ T-cells from HD (9.75%±1.56 and 11.30%±0.70 respectively) and in CD4⁺ (7.73%±0.18) and CD8⁺ T-cells (9.48%±0.93) from LP (FIG. 3A).

The qPCR products were further used for the amplification of dye-labelled oligonucleotides specific for CP and allowed to assess the complementary determining region 3 (CDR3) length distribution. For each TCRVβ gene segment tested from CD4⁺ and CD8⁺ T-cells from HD or from CD4⁺ T-cells isolated from LP and LSA patients, CDR3β length distribution profiles displayed Gaussian-like curves, the hallmark of a polyclonal T-cell repertoire (FIG. 3B). In contrast, CDR3β distribution profiles of the TCRVβ gene segment were altered with oligoclonal expansions in blood CD8⁺ T-cells from all LP patients, as shown in FIG. 3B top panel, for LP #2 for a CDR3β size of 8 amino-acids (AA). Similarly, CDR3β distribution profiles of the TCRVβ6 gene segment were altered with oligoclonal expansions in blood CD8⁺ T-cells from all LSA patients, as shown for LSA #1 for a CDR3β size of 8 AA (FIG. 3B, bottom panel). We next determined the Vβ-Jβ rearrangements corresponding to the observed oligoclonal expansions (FIG. 4), and further cloned and sequenced them. As shown in Table 2 and Table 3, one or two dominant CDR3β sequences were detected in each patient. Overall, neither CDR3β length nor consensus sequence was shared across all patients. However, all detected T CD8⁺ oligoclonal expansions for LP patients were using the TCRVβ gene segment, even in patients not sharing common class I HLA alleles (Table 1). Notably, 7 out of the 10 patients displayed Vβ3-Jβ2.7 clonotypic TCR (Table 3), a hallmark that we also previously revealed in 5 out of 10 LP patients with erosive oral form of LP (Viguier et al., 2015). Interestingly, the Vβ3-Jβ2.7 SxxxxYEQ CDR3 consensus sequence of 8 AA length was found in 5 out of the 20 studied patients with LP (LP here and erosive oral LP in (Viguier et al., 2015)). Regarding LSA patients, 4 out of the 10 patients displayed Vβ6-Jβ1.1 clonotypic TCR (Table 4), but neither CDR3β length nor CDR3 AA sequences were found similar in the different patients.

Overall, this set of data extends previous observations that LP patients with non-erosive and erosive oral forms display CD8⁺ T-cells with a skewed TCR repertoire towards the usage of a restricted set of TCRVβ segments including TCRVβ (Gotoh et al., 2008; Viguier et al., 2015). Interestingly, patients with another cutaneous and mucosal dermatosis such as LSA did not exhibit a skewed TCRVβ CTL repertoire.

### Example 10. CD8⁺ T-Cells Expressing Oligoclonal TCRVβ3⁺ in NELP Patients are Specific for HPV16 and Found in LP Lesions

An increased presence of TCRVβ positive cells has been reported in the mucosal T-cell infiltrates of LP patients (Simark-Mattsson et al., 1994). Moreover, intra-lesional CTL from LP patients are suspected to recognize an antigen associated with MHC class I molecule on lesional keratinocytes (Sugerman et al., 2000). Interestingly, we found high expression of cytotoxic molecules in blood CD8+ T cells of LP patients (FIG. 2), suggesting a more common phenomenon as observed in patients with chronic viral infections (Casazza et al., 2001). Several studies have shown the presence of several HPV genotypes in mucosal LP lesions, with a strong association with HPV16 genotype (Jontell et al., 1990; Miller et al., 1993; Lodi et al., 2005; Yildirim et al., 2011; Ma et al., 2016). In this context, we previously showed that Vβ3 TCR clonotypes in the blood and in the lesions of erosive oral LP patients were enriched in HPV16-specific CD8⁺ T-cells (Viguier et al., 2015). We assessed whether a similar observation could be made in LP patients with other forms than the erosive oral one. Therefore, we first assessed current and past HPV16 infection status of the studied patients. Detection of the various HPV genotypes in LP and LSA skin and/or mucosal lesions sampled at the time of the diagnosis was performed in all patients and HPV16 DNA was not found in any patient. Nevertheless, we detected capsid L1-specific IgG in sera of 9 out of 10 LP patients and of 7 out of 10 LSA patients (FIG. 5A). Expression of the oncoprotein E6, testifying of an active infection with HPV16, was assessed by detection of specific IgG in the sera and found only in LP patients (3 out of 10 patients), all with oral mucosal lesions of LP and associated capsid L1-Ig and without associated HPV-driven cancer (FIG. 5A). Since less than 1% of healthy controls harbor E6-specific IgG, the high prevalence in LP patients is in favor of a relevant HPV16 infection (Lang Kuhs et al., 2015).

Further, to test whether TCRVβ3⁺ clonal expansions exhibit HPV16-specificity, we stained PBMC from 3 HLA-A*0201⁺ LP patients of the cohort with peptide-containing MHC class I dextramer. As expected, almost no TCRVβ3⁺ T-cells were stained with HLA-A2/HIV-p17 Gag dextramer in these patients who are HIV negative (FIG. 5B). In contrast, a distinct population of CD8⁺ T-cells expressing a TCRVβ specific for the E7₁₃₋₂₄ immunodominant peptide of HPV16 was detected in the 3 tested patients, as shown for LP #10 (FIG. 5B), while almost none was detected in the 4 HLA-A*0201⁺ LSA patients (FIG. 5C). We then sorted out HPV16-specific CD8⁺ TCRVβ3⁺ cells as well as their CD8⁺ TCRVβ3⁺ Dext-HPV16^{neg} counterparts from LP #10 and LP #5. Spectratyping analysis and CDR3β sequencing were performed, revealing an enrichment of Dext-HPV16⁺ populations with the SLWAGNLREQ clonotype for LP #10 and the SLSTPNYEQ clonotype for LP #5 (FIG. 5D). In addition, in situ immunostaining analysis of LP lesional skin biopsy from LP #1 was performed. We found CD8⁺ T-cells bearing TCR specific for HLA-A2/HPV16 E7₁₃₋₂₄ peptide, as revealed by specific Dext-HPV16 staining, whereas staining with the irrelevant Dext-HIV yielded negative results (FIG. 5E). Finally, spectratyping analysis on tissue lesions from LP #3 revealed the infiltrate of T-cells bearing Vβ3-Jβ2.7 of 8 AA, as observed in the blood of this patient, as well as the presence of the SLRGAYEQ clonotype (FIG. 5F).

Since CTL from LSA patient blood displayed activated phenotype and biased TCR repertoire towards the Vβ6+ gene segment, we tested whether these CTL could also be specific to a distinct virus. It was shown that some EBV-specific CTL express TCR using Vβ6 gene segment. Thus, we tested whether PBMC from the HLA-A*0201+ LSA patients of the cohort could be stained with peptide-containing MHC class I dextramer. We detected a distinct population of CD8+ T-cells specific for the BMLF1 immunodominant peptide of EBV while, as expected, no T-cells were stained with HLA-A2/HIV-p17 Gag dextramer in these patients who are HIV negative (FIG. 7a). Then, EBV-specific CD8+ T cells as well as their CD8+ Dext-EBV− counterparts from LSA #1 and LSA #7 were sorted out. In contrast to what observed in NELP patients, clonotypes enriched in the blood of LSA patients were not found in Dext-EBV+ T-cells while still found in Dext-EBV− T-cells (FIG. 7b), suggesting that the enriched clonotypes found int the blood of LSA pateints are not EBV-specific.

TABLE 1
Characteristics of patients with LP at the time of the study

| | | | | Lichen | | | | |
|---|---|---|---|---|---|---|---|---|
| | | Duration of | | localization | | Current | | |
| | | Lichen | LP clinical | (BSA, if | Clinical status | treatment | Biopsy for | HLA-A2 |
| Patients | Sex/age | (years) | variant | applicable) | at inclusion | for lichen | research use | typing |
| NELP#1 | F/55 | 6 | Papular LP | Skin (45%) | Flare | UVB | Skin biopsy | + |
| NELP#2 | F/43 | 2 | Papular LP | Skin (2%) | Partial remission | TS | ND | − |
| NELP#3 | M/50 | 36 | Bullous, | Skin (10%), oral | Flare | None | Skin biopsy | − |
| | | | hypertrophic, oral | mucosa | | | | |
| | | | LP | | | | | |
| NELP#4 | M/50 | 10 | Non-erosive oral | Oral mucosa | Flare | MTX 15 mg/w. | ND | − |
| | | | LP | | | | | |
| NELP#5 | F/53 | 29 | Non-erosive oral | Oral and genital | Flare | Prednisone 10 | ND | + |
| | | | LP | mucosa | | mg/d. | | |
| NELP#6 | F/39 | 2 | Lichen | Scalp (5%), oral | Flare | TS, prednisone 4 | ND | + |
| | | | planopilaris | mucosa | | mg/d., | | |
| | | | and oral LP | | | ciclosporin 5 | | |
| | | | | | | mg/kg/d. | | |
| NELP#7 | F/51 | First | Papular LP | Skin (4%), oral | Flare | TS, acitretin | Skin biopsy | − |
| | | flare | | mucosa | | | | |
| NELP#8 | F/61 | 6 | Non-erosive oral | Oral mucosa | Partial remission | None | ND | − |
| | | | LP | | | | | |
| NELP#9 | F/55 | 2 | Lichen | Scalp (2%) | Flare | Topical | ND | − |
| | | | planopilaris | | | tacrolimus | | |
| NELP#10 | F/51 | 14 | Non-erosive oral | Oral mucosa | Flare | None | Oral mucosa | + |
| | | | LP | | | | biopsy | |

BSA: body surface area;
d: day;
F: female;
LP: lichen planus;
M: male;
MTX: methotrexate;
ND: not done;
TS: topical steroids;
UVB: phototherapy with ultraviolets B;
w: weeks

TABLE 2
Characteristics of patients with LSA at the time of the study

| | | Age at | | Current | HLA-A2 |
|---|---|---|---|---|---|
| Patients | Sex/age | onset | localization | treatment | typing |
| LSA#1 | F/73 | 58 | Vulva | TS | + |
| LSA#2 | F/76 | 74 | Vulva | None | − |
| LSA#3 | F/64 | 58 | Vulva | TS | − |
| LSA#4 | F/69 | 65 | Vulva | TS | + |
| LSA#5 | M/71 | 67 | Penis | TS | + |
| LSA#6 | M/28 | 25 | Penis | TS | − |
| LSA#7 | M/56 | 55 | Glans | TS, MTX | + |
| LSA#8 | F/70 | 54 | Vulva | TS | − |
| LSA#9 | M/72 | 69 | Glans | TS | − |
| LSA#10 | F/68 | 48 | Vulva | TS | − |

TS: topical steroids;
MTX: methotrexate

TABLE 3
CDR3β sequences in PBMC from LP patients
a) For each pool, 22-52 bacterial clones were sequenced.
b) Sequence occurrence/total number of sequences performed, shown as a percentage number.

| | CDR3β (nucleotide sequence) | | Deduced CDR3 | CDR3 | Freq |
|---|---|---|---|---|---|
| | (SEQ ID NOs: 13-24) | Jβ | (SEQ ID NOs: 1-12) | length | (%)^{b} |
| NELP#1 | AGTCGGACGGTAAACACCATA | 1.3 | SRTVNTI | 7 | 29.6 |
| | AGTTTGGTTGAAACAGGTTTTATAGGGACTACGTTT | 2.7 | SLVETGFIGTTFEVEQ | 16 | 11.1 |
| | GAGGTTGAGCAG | | | | |
| NELP#2 | AGTTTGGGTGTGGGCTACGAGCAG | 2.7 | SLGVGYEQ | 8 | 37.5 |
| NELP#3 | AGTCTACGGGGGGCCTACGAGCAG | 2.7 | SLRGAYEQ | 8 | 30.5 |
| NELP#4 | AGTTTTCAGGGGTTCGAGACCCAG | 2.5 | SFQGFETQ | 8 | 14.3 |
| NELP#5 | AGTTTATCGACTCCTAACTACGAGCAG | 2.7 | SLSTPNYEQ | 9 | 100 |
| NELP#6 | AGTTTTCAGGGGTACTACGAGCAG | 2.7 | SFQGYYEQ | 8 | 4 |
| NELP#7 | AGTTGGACTAGGACTTACAATGAGCAG | 2.1 | SWTRTYNEQ | 9 | 8 |
| NELP#8 | AGTTTTGGTCTTCGAGCGGGGACCTACGAGCAG | 2.7 | SFGLRAGTYEQ | 11 | 54.5 |
| NELP#9 | AGTTTGACTAGCGCCACCGGGGAGCTG | 2.2 | SLTSATGEL | 9 | 14.8 |
| NELP#10 | AGCCTCCGGGGGGCTATTAACGGGGAGCTG | 2.2 | SLRGAINGEL | 10 | 34.1 |
| | AGTTTATGGGCAGGGAATTTACGTGAGCAG | 2.7 | SLWAGNLREQ | 10 | 17.1 |

TABLE 4
Vβ6 CDR3β sequences in PBMC of LSA patients
a) For each pool. 18-43 bacterial clones were sequenced.
b) Sequence occurrence/total number of sequences performed. shown as a percentage number.

| | CDR3β (nucleotide sequence) | | Deduced CDR3 | CDR3 | Freq |
|---|---|---|---|---|---|
| | (SEQ ID NOs: 36-47) | Jβ | (SEQ ID NOs: 48-59) | length | (%)^{b} |
| LSA#1 | AGCTCCATGGGGGTCACTGAAGCT | 1.1 | SSMGVTEA | 8 | 72.7 |
| LSA#2 | AGCTTTTGGCAGGTCCCCGGGGAGCTG | 2.2 | SFWQVPGEL | 9 | 55.9 |
| LSA#3 | AGCCCATACAGGAGCTCCTACAATGAGCAG | 2.1 | SPYRSSYNEQ | 10 | 66.7 |
| LSA#4 | AGCTCGACTAGCTGGGGAGAGACCCAG | 2.5 | SSTSWGETQ | 9 | 33.4 |
| | AGCTTAGGCCGAGGCCCCGGAGATACGCAG | 2.3 | SLGRGPGDTQ | 10 | 48.5 |
| LSA#5 | AGCCACCAAACGGATGAAGCT | 1.1 | SHQTDEA | 7 | 17.9 |
| | AGCACTAGGACAGGGCGGTTTGAAAAACTG | 1.4 | STRTGRFEKL | 10 | 17.9 |
| LSA#6 | AGCTCAGCGGGACAGGGCTACACAGAAGCT | 1.1 | SSAGQGYTEA | 10 | 57 |
| LSA#7 | AGCTCACCGACAGGGGGCTTGGATGAAGCT | 1.1 | SSPTGGLDEAFF | 12 | 33.3 |
| LSA#8 | AGCCCCCCCACGGGCGACTACGAGCAG | 2.7 | SPPTGDYEQYF | 11 | 22.7 |
| LSA#9 | AGCTCCAATACGGCAAGCTCCTACAATGAGCAG | 2.1 | SSNTASSYNEQ | 11 | 50 |
| LSA#10 | AGCAATTCCACGTCGGGCTATGGCTAC | 1.2 | SNSTSGYGY | 9 | 40 |

### REFERENCES

Alaizari, N. A., Al-Maweri, S. A., Al-Shamiri, H. M., Tarakji, B., and Shugaa-Addin, B. (2016). Hepatitis C virus infections in oral lichen planus: a systematic review and meta-analysis. Aust Dent J 61, 282-287.
Andrei, G., Topalis, D., De Schutter, T., and Snoeck, R. (2015). Insights into the mechanism of action of cidofovir and other acyclic nucleoside phosphonates against polyoma- and papillomaviruses and non-viral induced neoplasia. Antiviral Res 114, 21-46.
Birkenfeld, S., Dreiher, J., Weitzman, D., and Cohen, A. D. (2011). A study on the association with hepatitis B and hepatitis C in 1557 patients with lichen planus. J Eur Acad Dermatol Venereol 25, 436-440.
Calista, D., and Morri, M. (2004). Lichen planus induced by hepatitis B vaccination: a new case and review of the literature. Int J Dermatol 43, 562-564.
Casazza, J. P., Betts, M. R., Picker, L. J., and Koup, R. A. (2001). Decay kinetics of human immunodeficiency virus-specific CD8+ T cells in peripheral blood after initiation of highly active antiretroviral therapy. J Virol 75, 6508-6516.
Ciaccio, M., and Rebora, A. (1990). Lichen planus following HBV vaccination: a coincidence? Br J Dermatol 122, 424.
Clifford, G. M., Vaccarella, S., Franceschi, S., Tenet, V., Umulisa, M. C., Tshomo, U., Dondog, B., Vorsters, A., Tommasino, M., Heideman, D. A., Snijders, P. J., and Gheit, T. (2016). Comparison of Two Widely Used Human Papillomavirus Detection and Genotyping Methods, GP5+/6+-Based PCR Followed by Reverse Line Blot Hybridization and Multiplex Type-Specific E7-Based PCR. J Clin Microbiol 54, 2031-2038.
Combita, A. L., Bravo, M. M., Touze, A., Orozco, O., and Coursaget, P. (2002). Serologic response to human oncogenic papillomavirus types 16, 18, 31, 33, 39, 58 and 59 virus-like particles in colombian women with invasive cervical cancer. Int J Cancer 97, 796-803.
Fazilleau, N., Bachelez, H., Gougeon, M. L., and Viguier, M. (2007). Cutting edge: size and diversity of CD4+CD25high Foxp3+ regulatory T cell repertoire in humans: evidence for similarities and partial overlapping with CD4+CD25− T cells. J Immunol 179, 3412-3416.
Gheit, T., Landi, S., Gemignani, F., Snijders, P. J., Vaccarella, S., Franceschi, S., Canzian, F., and Tommasino, M. (2006). Development of a sensitive and specific assay combining multiplex PCR and DNA microarray primer extension to detect high-risk mucosal human papillomavirus types. J Clin Microbiol 44, 2025-2031.
Gotoh, A., Hamada, Y., Shiobara, N., Kumagai, K., Seto, K., Horikawa, T., and Suzuki, R. (2008). Skew in T cell receptor usage with polyclonal expansion in lesions of oral lichen planus without hepatitis C virus infection. Clin Exp Immunol 154, 192-201.
Jayavelu, P., and Sambandan, T. (2012). Prevalence of hepatitis C and hepatitis B virus infection(s) in patients with oral lichen planus. J Pharm Bioallied Sci 4, S397-405.
Jontell, M., Watts, S., Wallstrom, M., Levin, L., and Sloberg, K. (1990). Human papilloma virus in erosive oral lichen planus. Journal of oral pathology & medicine: official publication of the International Association of Oral Pathologists and the American Academy of Oral Pathology 19, 273-277.
Lang Kuhs, K. A., Anantharaman, D., Waterboer, T., Johansson, M., Brennan, P., Michel, A., Willhauck-Fleckenstein, M., Purdue, M. P., Holcatova, I., Ahrens, W., Lagiou, P., Polesel, J., Simonato, L., Merletti, F., Healy, C. M., Kjaerheim, K., Conway, D. I., Macfarlane, T. V., Thomson, P., Castellsague, X., Znaor, A., Black, A., Huang, W. Y., Krogh, V., Trichopoulou, A., Bueno-De-Mesquita, H. B., Clavel-Chapelon, F., Weiderpass, E., Ekstrom, J., Riboli, E., Tjonneland, A., Sanchez, M. J., Travis, R. C., Hildesheim, A., Pawlita, M., and Kreimer, A. R. (2015). Human Papillomavirus 16 E6 Antibodies in Individuals without Diagnosed Cancer: A Pooled Analysis. Cancer Epidemiol Biomarkers Prev 24, 683-689.
Le Cleach, L., and Chosidow, O. (2012). Clinical practice. Lichen planus. N Engl J Med 366, 723-732.
Lodi, G., Franchini, R., Bez, C., Sardella, A., Moneghini, L., Pellegrini, C., Bosari, S., Manfredi, M., Vescovi, P., and Carrassi, A. (2010). Detection of survivin mRNA in healthy oral mucosa, oral leucoplakia and oral cancer. Oral Dis 16, 61-67.
Lodi, G., Scully, C., Carrozzo, M., Griffiths, M., Sugerman, P. B., and Thongprasom, K. (2005). Current controversies in oral lichen planus: report of an international consensus meeting. Part 1. Viral infections and etiopathogenesis. Oral Surg Oral Med Oral Pathol Oral Radiol Endod 100, 40-51.
Ma, J., Zhang, J., Zhang, Y., Lv, T., and Liu, J. (2016). The Magnitude of the Association between Human Papillomavirus and Oral Lichen Planus: A Meta-Analysis. PLoS One 11, e0161339.
Mattila, R., Rautava, J., and Syrjanen, S. (2012). Human papillomavirus in oral atrophic lichen planus lesions. Oral Oncol 48, 980-984.
Miller, C. S., White, D. K., and Royse, D. D. (1993). In situ hybridization analysis of human papillomavirus in orofacial lesions using a consensus biotinylated probe. Am J Dermatopathol 15, 256-259.
Nagao, Y., Kawahigashi, Y., Kimura, K., and Sata, M. (2017). Effect of Oral Care Gel for Burning Mouth Syndrome in a Patient with Hepatitis C: A Case Report. Case Rep Gastroenterol 11, 480-487.
Natale, C., Giannini, T., Lucchese, A., and Kanduc, D. (2000). Computer-assisted analysis of molecular mimicry between human papillomavirus 16 E7 oncoprotein and human protein sequences. Immunol Cell Biol 78, 580-585.
Rebora, A., and Rongioletti, F. (1984). Lichen planus and chronic active hepatitis. A retrospective survey. Acta Derm Venereol 64, 52-56.
Schmitt, M., Dondog, B., Waterboer, T., Pawlita, M., Tommasino, M., and Gheit, T. (2010). Abundance of multiple high-risk human papillomavirus (HPV) infections found in cervical cells analyzed by use of an ultrasensitive HPV genotyping assay. J Cin Microbiol 48, 143-149.
Selmi, C., Leung, P. S., Sherr, D. H., Diaz, M., Nyland, J. F., Monestier, M., Rose, N. R., and Gershwin, M. E. (2012). Mechanisms of environmental influence on human autoimmunity: a National Institute of Environmental Health Sciences expert panel workshop. J Autoimmun 39, 272-284.
Shang, Q., Peng, J., Zhou, Y., Chen, Q., and Xu, H. (2020). Association of Human Papillomavirus With Oral Lichen Planus and Oral Leukoplakia: A Meta-analysis. J Evid Based Dent Pract 20, 101485.
Shengyuan, L., Songpo, Y., Wen, W., Wenjing, T., Haitao, Z., and Binyou, W. (2009). Hepatitis C virus and lichen planus: a reciprocal association determined by a meta-analysis. Arch Dermatol 145, 1040-1047.
Simark-Mattsson, C., Bergenholtz, G., Jontell, M., Tarkowski, A., and Dahlgren, U. I. (1994). T cell receptor V-gene usage in oral lichen planus; increased frequency of T cell receptors expressing V alpha 2 and V beta 3. Clin Exp Immunol 98, 503-507.
Song, J., Zhang, Z., Ji, X., Su, S., Liu, X., Xu, S., Han, Y., Mu, D., and Liu, H. (2016). Lack of evidence of hepatitis in patients with oral lichen planus in China: A case control study. Med Oral Patol Oral Cir Bucal 21, e161-168.
Sugerman, P. B., Satterwhite, K., and Bigby, M. (2000). Autocytotoxic T-cell clones in lichen planus. The British journal of dermatology 142, 449-456.
Viguier, M., Bachelez, H., Poirier, B., Kagan, J., Battistella, M., Aubin, F., Touze, A., Carmagnat, M., Frances, C., Gougeon, M. L., and Fazilleau, N. (2015). Peripheral and Local Human Papillomavirus 16-Specific CD8(+) T-Cell Expansions Characterize Erosive Oral Lichen Planus. Journal of Investigative Dermatology 135, 418-424.
Wenzel, J., Scheler, M., Proelss, J., Bieber, T., and Tuting, T. (2006). Type I interferon-associated cytotoxic inflammation in lichen planus. J Cutan Pathol 33, 672-678.
Yildirim, B., Senguven, B., and Demir, C. (2011). Prevalence of herpes simplex, Epstein Barr and human papilloma viruses in oral lichen planus. Med Oral Patol Oral Cir Bucal 16, e170-174.
Zhou, X. J., Sugerman, P. B., Savage, N. W., Walsh, L. J., and Seymour, G. J. (2002). Intra-epithelial CD8+ T cells and basement membrane disruption in oral lichen planus. Journal of oral pathology & medicine: official publication of the International Association of Oral Pathologists and the American Academy of Oral Pathology 31, 23-27.

We claim:

1. A method for detecting the presence of an immune response against a human papilloma virus infection in a biological sample of a patient suspected to have non-erosive lichen planus (NELP), said method comprising:
isolating T cells from a biological sample from the patient; and
detecting expansion of a clonal population of CD8+ TCRVbeta3+ T cells recognizing the HPV16 E713-24 epitope in the sample,
wherein said clonal population has a single CDR3beta sequence in the TCRVbeta3 gene segment.

2. The method of claim 1, wherein the CDR3beta sequence in the TCRVbeta3 gene segment in the CD8+ T cells encodes any one of the following peptides: SRTVNTI (SEQ ID NO:1), SLVETGFIGTTFEVEQ (SEQ ID NO:2), SLGVGYEQ (SEQ ID NO:3), SLRGAYEQ (SEQ ID NO:4), SFQGFETQ (SEQ ID NO:5), SLSTPNYEQ (SEQ ID NO:6), SFQGYYEQ (SEQ ID NO:7), SWTRTYNEQ (SEQ ID NO:8), SFGLRAGTYEQ (SEQ ID NO:9), SLTSATGEL (SEQ ID NO:10), SLRGAINGEL (SEQ ID NO:11), SLWAGNLREQ (SEQ ID NO:12), a consensus peptide consisting of: S in position 1, L, V or F in position 2, G or R in position 3, V or G in position 4, G, A or H in position 5, Y in position 6, E in position 7, and Q in position 8 (SEQ ID NO:60).

3. The method of claim 1, further comprising staining PMBC of the biological sample from the patient with peptide-containing MHC class I dextramer specific for E713-24 immunodominant peptide of HPV16.

4. The method of claim 1, further comprising spectratyping analysis of the biological sample from the patient.

5. The method of claim 1, wherein the patient is suspected to have a skin lichen planus.

6. The method of claim 1, wherein the patient is suspected to have a scalp lichen planus.

7. The method of claim 1, wherein the patient is suspected to have a nail lichen planus.

8. A method for detecting the presence of a clonal expansion of CD8+ T cells in a biological sample of a patient suspected to have a lichen sclerosus et atrophicus (LSA), said method comprising:
isolating T cells from a biological sample from the patient; and
detecting expansion of a clonal population of CD8+ TCRVbeta6+ T cells in the sample,
wherein said clonal population has a single CDR3beta sequence in the TCRVbeta6 gene segment.

9. The method of claim 8, wherein the CDR3beta sequence in the TCRVbeta6 gene segment in the CD8+ T cells encodes any one of the following peptides: SSMGVTEASEQ (SEQ ID NO: 48), SFWQVPGEL (SEQ ID NO: 49), SPYRSSYNEQ (SEQ ID NO: 50), SSTSWGETQ (SEQ ID NO: 51), SLGRGPGDTQ (SEQ ID NO: 52), SHQTDEA (SEQ ID NO: 53), STRTGRFEKL (SEQ ID NO: 54), SSAGQGYTEA (SEQ ID NO: 55), SSPTGGLDEAFF (SEQ ID NO: 56), SPPTGDYEQYF (SEQ ID NO: 57), SSNTASSYNEQ (SEQ ID NO: 58), SNSTSGYGY (SEQ ID NO: 59).

10. The method of any of claims 1 to 9, wherein the biological sample is a sample of blood, or of a lesional biopsy, more particularly a lesional biopsy of skin, nail, or scalp.

11. A method for detecting an HPV infection in a patient suspected to have non-erosive lichen planus (NELP) comprising providing a non-mucosal cell sample from the patient, contacting the non-mucosal sample with at least one HPV peptide, and detecting the interaction between the HPV peptide and cells in the sample.

12. The method of claim 11, wherein the HPV peptide comprises the amino acid sequence YMLDLQPETT (SEQ ID NO:33).

13. The method of claim 11, wherein the non-mucosal sample is a skin sample.

14. The method of claim 11, wherein the non-mucosal sample is a scalp sample.

15. The method of claim 11, wherein the non-mucosal sample is a nail sample.

16. A method for diagnosing in a patient an LP, preferably an LP clinical form other than the erosive oral form of LP, comprising detecting TCRVβ3+ CD8+ T-cells oligoclonal expansion in a sample of the peripheral blood or of a lesion of the patient to be diagnosed.

17. The method according to claim 16, wherein said CD8+ T-cells exhibit a cytotoxic phenotype.

18. The method according to claim 17, wherein said oligoclonal expansions can be detected by the curve of the CDR3beta length distribution of CD8+ Vβ3-Cβ rearrangement.

19. A method for diagnosing in a patient an LP, preferably an LP clinical form other than the erosive oral form of LP, comprising detecting a predominant usage of the TCRVβ3 gene segment in a sample of circulating CD8+ T cells of the patient with respect to the same usage in circulating CD4+ T-cells counterparts.

20. A method for diagnosing in a patient an LP, preferably an LP clinical form other than the erosive oral form of LP, comprising detecting oligoclonal CD8+ T cells specific for HPV16, preferably CD8+ Vβ3+ T cells specific for HPV16, in a blood sample of the patient, and/or in a sample from LP lesions of the patient.

21. The method according to claim 20, wherein detecting oligoclonal CD8+ T cells specific for HPV16 is made by flow cytometry.

22. The method for diagnosing according to any one of claims 16 to 21, allowing a differential diagnosis of LP versus other cutaneous or mucosal dermatosis.

23. The method according to any one of claims 16 to 22, wherein said method allows to discriminate between LP and another cutaneous or mucosal dermatosis, which is not an LP, especially LSA, lichenoid GVHD, mucosal pemphigoid, psoriasis, and Brocq's pseudo alopecia.

24. The method according to any one of claims 16 to 23, for diagnosing a NELP.

25. The method of any of claims 16 to 24, wherein the biological sample is a sample of blood, or of a lesional biopsy, more particularly a lesional biopsy of skin, nail, genital organ or scalp.

26. A kit comprising:
an antibody recognizing T-cells,
an antibody recognizing CD8+ T cells,
an antibody recognizing CD4+ T cells, and
an antibody recognizing TCRVβ3+ T cells.

27. The kit according to claim 26, wherein said antibody recognizing T-cells, is an anti-CD3 antibody.

28. The kit according to claim 26, wherein said antibody recognizing CD8+ T cells is an anti-CD8 antibody.

29. The kit according to claim 26, wherein said antibody recognizing CD4+ T cells is an anti-CD4 antibody.

30. The kit according to claim 26, wherein said antibody recognizing TCRVβ3+ T cells is an anti-TCRVβ3 antibody.

31. The kit according to any one of claims 26 to 31, further comprising a dextramer-HPV16.

32. A method for treating a patient suffering from a LP, preferably an LP clinical form other than the erosive oral form of LP, comprising the steps of:
immunotargeting the CD8+ TCRVβ3+ T-cell population of said patient, and
deleting or anergizing said T-cell population,
thus treating the LP.

33. The method of claim 32, wherein said LP is a NELP.

34. A method for monitoring treatment of an LP patient, comprising determining in a biological sample obtained from said patient, the usage of the TCRVβ3 gene segment in circulating CD8+ T cells, before and after treatment, and detecting a modulation or absence of modulation of the TCRVβ3 gene segment usage, preferably a decrease of said usage.

35. A method for monitoring treatment of an LP patient, comprising assaying oligoclonal CD8+ T cells specific for HPV16 in a biological sample obtained from the patient, before and after treatment, and detecting a decrease of the number of CD8+ T cells specific for HPV16, preferably of the number of CD8+ Vβ3+ T cells specific for HPV16.

36. The methods according to claim 34 or 35, wherein the LP patient is an NELP patient, or a patient suffering from a non-oral LP.


SEQUENCE LISTING (60 sequences)

SEQ ID NO: 1 - protein, 7 aa, Homo sapiens
SRTVNTI

SEQ ID NO: 2 - protein, 16 aa, Homo sapiens
SLVETGFIGTTFEVEQ

SEQ ID NO: 3 - protein, 8 aa, Homo sapiens
SLGVGYEQ

SEQ ID NO: 4 - protein, 8 aa, Homo sapiens
SLRGAYEQ

SEQ ID NO: 5 - protein, 8 aa, Homo sapiens
SFQGFETQ

SEQ ID NO: 6 - protein, 9 aa, Homo sapiens
SLSTPNYEQ

SEQ ID NO: 7 - protein, 8 aa, Homo sapiens
SFQGYYEQ

SEQ ID NO: 8 - protein, 9 aa, Homo sapiens
SWTRTYNEQ

SEQ ID NO: 9 - protein, 11 aa, Homo sapiens
SFGLRAGTYEQ

SEQ ID NO: 10 - protein, 9 aa, Homo sapiens
SLTSATGEL

SEQ ID NO: 11 - protein, 10 aa, Homo sapiens
SLRGAINGEL

SEQ ID NO: 12 - protein, 10 aa, Homo sapiens
SLWAGNLREQ

SEQ ID NO: 13 - DNA, 21 bp, Homo sapiens
  source 1..21: mol_type = genomic DNA
agtcggacggtaaacaccata

SEQ ID NO: 14 - DNA, 48 bp, Homo sapiens
  source 1..48: mol_type = genomic DNA
agtttggttgaaacaggttttatagggactacgtttgaggttgagcag

SEQ ID NO: 15 - DNA, 24 bp, Homo sapiens
  source 1..24: mol_type = genomic DNA
agtttgggtgtgggctacgagcag

SEQ ID NO: 16 - DNA, 24 bp, Homo sapiens
  source 1..24: mol_type = genomic DNA
agtctacggggggcctacgagcag

SEQ ID NO: 17 - DNA, 24 bp, Homo sapiens
  source 1..24: mol_type = genomic DNA
agttttcaggggttcgagacccag

SEQ ID NO: 18 - DNA, 27 bp, Homo sapiens
  source 1..27: mol_type = genomic DNA
agtttatcgactcctaactacgagcag

SEQ ID NO: 19 - DNA, 24 bp, Homo sapiens
  source 1..24: mol_type = genomic DNA
agttttcaggggtactacgagcag

SEQ ID NO: 20 - DNA, 27 bp, Homo sapiens
  source 1..27: mol_type = genomic DNA
agttggactaggacttacaatgagcag

SEQ ID NO: 21 - DNA, 33 bp, Homo sapiens
  source 1..33: mol_type = genomic DNA
agttttggtcttcgagcggggacctacgagcag

SEQ ID NO: 22 - DNA, 27 bp, Homo sapiens
  source 1..27: mol_type = genomic DNA
agtttgactagcgccaccggggagctg

SEQ ID NO: 23 - DNA, 30 bp, Homo sapiens
  source 1..30: mol_type = genomic DNA
agcctccggggggctattaacggggagctg

SEQ ID NO: 24 - DNA, 30 bp, Homo sapiens
  source 1..30: mol_type = genomic DNA
agtttatgggcagggaatttacgtgagcag

SEQ ID NO: 25 - protein, 9 aa, Human papilloma virus
HYNIVTFCC

SEQ ID NO: 26 - protein, 9 aa, Human papilloma virus
KLCLRFLSK

SEQ ID NO: 27 - protein, 9 aa, Human papilloma virus
KPTLKEYVL

SEQ ID NO: 28 - protein, 10 aa, Human papilloma virus
LLMGTLGIVC

SEQ ID NO: 29 - protein, 9 aa, Human papilloma virus
MLDLQPETT

SEQ ID NO: 30 - protein, 9 aa, Human papilloma virus
NTLEQTVKK

SEQ ID NO: 31 - protein, 9 aa, Human papilloma virus
RAHYNIVTF

SEQ ID NO: 32 - protein, 9 aa, Human papilloma virus
VPTLQDVVL

SEQ ID NO: 33 - protein, 10 aa, Human papilloma virus
YMLDLQPETT

SEQ ID NO: 34-35 - skipped (2 entries)

SEQ ID NO: 36 - DNA, 24 bp, Homo sapiens
  source 1..24: mol_type = genomic DNA
agctccatgggggtcactgaagct

SEQ ID NO: 37 - DNA, 27 bp, Homo sapiens
  source 1..27: mol_type = genomic DNA
agcttttggcaggtccccggggagctg

SEQ ID NO: 38 - DNA, 30 bp, Homo sapiens
  source 1..30: mol_type = genomic DNA
agcccatacaggagctcctacaatgagcag

SEQ ID NO: 39 - DNA, 27 bp, Homo sapiens
  source 1..27: mol_type = genomic DNA
agctcgactagctggggagagacccag

SEQ ID NO: 40 - DNA, 30 bp, Homo sapiens
  source 1..30: mol_type = genomic DNA
agcttaggccgaggccccggagatacgcag

SEQ ID NO: 41 - DNA, 21 bp, Homo sapiens
  source 1..21: mol_type = genomic DNA
agccaccaaacggatgaagct

SEQ ID NO: 42 - DNA, 30 bp, Homo sapiens
  source 1..30: mol_type = genomic DNA
agcactaggacagggcggtttgaaaaactg

SEQ ID NO: 43 - DNA, 30 bp, Homo sapiens
  source 1..30: mol_type = genomic DNA
agctcagcgggacagggctacacagaagct

SEQ ID NO: 44 - DNA, 30 bp, Homo sapiens
  source 1..30: mol_type = genomic DNA
agctcaccgacagggggcttggatgaagct

SEQ ID NO: 45 - DNA, 27 bp, Homo sapiens
  source 1..27: mol_type = genomic DNA
agcccccccacgggcgactacgagcag

SEQ ID NO: 46 - DNA, 33 bp, Homo sapiens
  source 1..33: mol_type = genomic DNA
agctccaatacggcaagctcctacaatgagcag

SEQ ID NO: 47 - DNA, 27 bp, Homo sapiens
  source 1..27: mol_type = genomic DNA
agcaattccacgtcgggctatggctac

SEQ ID NO: 48 - protein, 8 aa, Homo sapiens
SSMGVTEA

SEQ ID NO: 49 - protein, 9 aa, Homo sapiens
SFWQVPGEL

SEQ ID NO: 50 - protein, 10 aa, Homo sapiens
SPYRSSYNEQ

SEQ ID NO: 51 - protein, 9 aa, Homo sapiens
SSTSWGETQ

SEQ ID NO: 52 - protein, 10 aa, Homo sapiens
SLGRGPGDTQ

SEQ ID NO: 53 - protein, 7 aa, Homo sapiens
SHQTDEA

SEQ ID NO: 54 - protein, 10 aa, Homo sapiens
STRTGRFEKL

SEQ ID NO: 55 - protein, 10 aa, Homo sapiens
SSAGQGYTEA

SEQ ID NO: 56 - protein, 12 aa, Homo sapiens
SSPTGGLDEAFF

SEQ ID NO: 57 - protein, 11 aa, Homo sapiens
SPPTGDYEQYF

SEQ ID NO: 58 - protein, 11 aa, Homo sapiens
SSNTASSYNEQ

SEQ ID NO: 59 - protein, 9 aa, Homo sapiens
SNSTSGYGY

SEQ ID NO: 60 - protein, 8 aa, synthetic construct
  VAR_SEQ 2: note = L, V or F
  VAR_SEQ 3: note = G or R
  VAR_SEQ 4: note = V or G
  VAR_SEQ 5: note = G, A or H
SXXXXYEQ